SEQUENCE LISTING (49 sequences)

SEQ ID NO: 1 - protein, 439 aa, synthetic construct
SYELTQPPSVSVSPGQTASITCGGNNIGSKGVHWYQQKPGQSPVLVISYDSDRPSGIPERFSGSNSGNTATLTISGTQAMDEADYYCQVWDSSTRVFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 2 - protein, 11 aa, synthetic construct
GGNNIGSKGVH

SEQ ID NO: 3 - protein, 7 aa, synthetic construct
YDSDRPS

SEQ ID NO: 4 - protein, 9 aa, synthetic construct
QVWDSSTRV

SEQ ID NO: 5 - protein, 106 aa, synthetic construct
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 6 - protein, 106 aa, synthetic construct
SYELTQPPSVSVSPGQTASITCGGNNIGSKGVHWYQQKPGQSPVLVISYDSDRPSGIPERFSGSNSGNTATLTISGTQAMDEADYYCQVWDSSTRVFGTGTKVTVL

SEQ ID NO: 7 - protein, 217 aa, synthetic construct
APEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 8 - protein, 223 aa, synthetic construct
EVQLVESGGGLVQPGGSLRLSCAASGFTFSSYAMHWVRQAPGKGLEWVAVISYDGSNKYYADSVKGRFTISRDNAKNSLYLQMNSLRAEDTAVYYCARDKGIGTSPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 9 - protein, 5 aa, synthetic construct
SYAMH

SEQ ID NO: 10 - protein, 7 aa, synthetic construct
GFTFSSY

SEQ ID NO: 11 - protein, 10 aa, synthetic construct
GFTFSSYAMH

SEQ ID NO: 12 - protein, 17 aa, synthetic construct
VISYDGSNKYYADSVKG

SEQ ID NO: 13 - protein, 6 aa, synthetic construct
SYDGSN

SEQ ID NO: 14 - protein, 11 aa, synthetic construct
DKGIGTSPLDY

SEQ ID NO: 15 - protein, 103 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 16 - protein, 120 aa, synthetic construct
EVQLVESGGGLVQPGGSLRLSCAASGFTFSSYAMHWVRQAPGKGLEWVAVISYDGSNKYYADSVKGRFTISRDNAKNSLYLQMNSLRAEDTAVYYCARDKGIGTSPLDYWGQGTLVTVSS

SEQ ID NO: 17 - protein, 217 aa, synthetic construct
QSVLTQPPSASGTPGQRVTISCTGSSSNIGSNTVNWYQQLPGTAPKLLIYSDNQRPSGVPDRFSGSTSGTSASLAISGLRSEDEADYFCATWEDVLAGYVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 18 - protein, 13 aa, synthetic construct
TGSSSNIGSNTVN

SEQ ID NO: 19 - protein, 7 aa, synthetic construct
SDNQRPS

SEQ ID NO: 20 - protein, 12 aa, synthetic construct
ATWEDVLAGYVV

SEQ ID NO: 21 - protein, 106 aa, synthetic construct; identical to SEQ ID NO: 5

SEQ ID NO: 22 - protein, 111 aa, synthetic construct
QSVLTQPPSASGTPGQRVTISCTGSSSNIGSNTVNWYQQLPGTAPKLLIYSDNQRPSGVPDRFSGSTSGTSASLAISGLRSEDEADYFCATWEDVLAGYVVFGGGTKLTVL

SEQ ID NO: 23 - protein, 449 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCWRWLNLGDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 5 aa, synthetic construct
SYAMS

SEQ ID NO: 25 - protein, 7 aa, synthetic construct
GFTFSSY

SEQ ID NO: 26 - protein, 10 aa, synthetic construct
GFTFSSYAMS

SEQ ID NO: 27 - protein, 17 aa, synthetic construct
AISGSGGSTYYADSVKG

SEQ ID NO: 28 - protein, 6 aa, synthetic construct
SGSGGS

SEQ ID NO: 29 - protein, 10 aa, synthetic construct
WLNLGDAFDI

SEQ ID NO: 30 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 119 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCWRWLNLGDAFDIWGQGTMVTVSS

SEQ ID NO: 32 - protein, 103 aa, synthetic construct; identical to SEQ ID NO: 15

SEQ ID NO: 33 - protein, 10 aa, synthetic construct
DKTHTCPPCP

SEQ ID NO: 34 - protein, 217 aa, synthetic construct
APEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 438 aa, synthetic construct
SYELTQPPSVSVSPGQTASITCGGNNIGSKGVHWYQQKPGQSPVLVISYDSDRPSGIPERFSGSNSGNTATLTISGTQAMDEADYYCQVWDSSTRVFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 36 - protein, 448 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCWRWLNLGDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 37 - protein, 212 aa, synthetic construct
SYELTQPPSVSVSPGQTASITCGGNNIGSKGVHWYQQKPGQSPVLVISYDSDRPSGIPERFSGSNSGNTATLTISGTQAMDEADYYCQVWDSSTRVFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 38 - protein, 450 aa, synthetic construct
EVQLVESGGGLVQPGGSLRLSCAASGFTFSSYAMHWVRQAPGKGLEWVAVISYDGSNKYYADSVKGRFTISRDNAKNSLYLQMNSLRAEDTAVYYCARDKGIGTSPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 449 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCWRWLNLGDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 212 aa, synthetic construct; identical to SEQ ID NO: 37

SEQ ID NO: 41 - protein, 450 aa, synthetic construct
EVQLVESGGGLVQPGGSLRLSCAASGFTFSSYAMHWVRQAPGKGLEWVAVISYDGSNKYYADSVKGRFTISRDNAKNSLYLQMNSLRAEDTAVYYCARDKGIGTSPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCRKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 42 - protein, 449 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCWRWLNLGDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCEKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCEVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 330 aa, Homo sapiens [sequence omitted]

SEQ ID NO: 44 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 45 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 46 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 47 - protein, 106 aa, Homo sapiens
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 48 - protein, 106 aa, Homo sapiens; identical to SEQ ID NO: 5

SEQ ID NO: 49 - protein, 106 aa, Homo sapiens; identical to SEQ ID NO: 5

(19) United States
(12) Patent Application Publication
(10) Pub. No.: US 2024/0360214 A1
(43) Pub. Date: Oct. 31, 2024

(54) LILRB1 and LILRB2 ANTIBODIES AND METHODS OF USE THEREOF

(71) Applicant: PFIZER INC., NEW YORK, NY (US)

(72) Inventors: James Reasoner APGAR, Newton, MA (US); Wei CAO, Lexington, MA (US); Timothy HEMESATH, Caldwell, NJ (US); Christina Lynn O'HALLORAN, Metuchen, NJ (US); Lan WU, Wayne, NJ (US); Ming ZHU, Short Hills, NJ (US)

(73) Assignee: PFIZER INC., NEW YORK, NY (US)

(21) Appl. No.: 18/639,118

(22) Filed: Apr. 18, 2024

Related U.S. Application Data
(60) Provisional application No. 63/460,598, filed on Apr. 19, 2023.

(51) Int. Cl. C07K 16/28; A61K 39/00; A61P 35/00

(52) U.S. Cl. CPC C07K 16/2803; A61P 35/00; C07K 16/2818; A61K 2039/505; A61K 2039/507; C07K 2317/31; C07K 2317/76; C07K 2317/92

(57) ABSTRACT

The invention is directed to Anti-LILRB1 and anti-LILRB2 bispecific antibodies, anti-LILRB1 antibodies, and anti-LILRB2 antibodies, their uses and pharmaceutical compositions thereof.

### CROSS-REFERENCE TO RELATED APPLICATIONS

This application claims priority, under 35 U.S.C. § 119 (e), to U.S. Provisional Application No. 63/460,598, filed Apr. 19, 2023, the disclosure of which is hereby incorporated by reference in its entirety.

### REFERENCE TO SEQUENCE LISTING

This application is being filed via Patent Center and includes an electronically submitted sequence listing in .xml format. The .xml file contains a sequence listing entitled “PC72966A_SeqListing_ST26.xml” created on Apr. 16, 2024 and have a file size of 50 Kb. The sequence listing contains in this .xml file is part of the specification and is herein incorporated by reference in its entirety.

### BACKGROUND

Leukocyte immunoglobulin-like receptor B1 (LILRB1) and B2 (LILRB2) are negative signaling immune receptors that are expressed on the surface of myeloid-derived suppressive cells (MDSCs), dendritic cells (DCs), monocytes and macrophages. LILRB1 is additionally present on subsets of T cells and NK cells, and most normal B cells. Human Leukocyte antigen G (HLA-G) is a non-classical MHC molecule that exists in soluble and membrane-inserted forms that binds LILRB1/2. HLA-G is expressed by fetal extravillous trophoblasts at the maternal-fetal interface and plays a critical role in successful pregnancy by down-regulating the maternal immune response to the allogeneic fetus.

LILRB1/2 ligation by classical and non-classical Class I MHC has been shown to drive multiple mechanisms of innate and adaptive immune down-modulation and consequent tumor survival. LILRB1/2 signaling inhibits allo-stimulation of CD4 T cells, NK and CD8 T cell cytolytic function, maturation and function of dendritic cells and monocytes, and the phagocytic activity of macrophages toward tumor cells.

HLA-G has been shown to be re-expressed by some solid tumors and tumor-infiltrating leukocytes (TILs). Expression has been associated with worse prognosis and refractory disease in numerous cancers, including but not limited to kidney, ovary, breast, lung and colon. It is believed that blocking LILRB1/B2 interaction with HLA with an antagonist antibody may limit myeloid/lymphoid-induced immune suppression and thereby promote anti-tumor immunity. There is a need to make therapeutic agents that specifically and simultaneously block LILRB1 and LILRB2 receptors for the treatment of solid tumors.

### SUMMARY OF THE INVENTION

The present disclosure provides bispecific antibodies that bind to LILRB1 and LILRB2, anti-LILRB1 antibodies that bind to LILRB1 and anti-LILRB2 antibodies that bind to LILRB2, as well as uses of the bispecific antibodies and antibodies and associated methods, and the processes for making, preparing, and producing these antibodies. Antibodies of the disclosure are useful in one or more of diagnosis, prophylaxis, or treatment of disorders or conditions mediated by, or associated with LILRB1 activity, LILRB2 activity or both, including, but not limited to cancer and especially solid tumors. The disclosure further encompasses expression of antibodies, and preparation and manufacture of compositions comprising antibodies of the disclosure, such as medicaments for the use of the antibodies.

Polynucleotides encoding antibodies that bind to LILRB1, LILRB2 or both, are provided. Polynucleotides encoding antibody heavy chains or light chains, or both are also provided. Host cells that express the antibodies are provided. Methods of treatment using the antibodies are provided. Such methods include, but are not limited to, one or more of methods of treating or methods of preventing diseases associated with or mediated by LILRB1 expression, LILRB2 expression, or both, or LILRB1 binding, or LILRB2 binding, or both, such as cancer and especially solid tumors.

### BRIEF DESCRIPTION OF THE DRAWINGS

FIG. 1A depicts that in Example 2, anti-LILRB1 antibody Ab-B1-1704 inhibited HLA-G binding to LILRB1 expressed on THP1 cells, while anti-LILRB2 antibody Ab-B2-1825 did not inhibit HLA-G binding to LILRB1 expressed on THP1 cells.

FIG. 1B depicts that in Example 2, anti-LILRB1 antibody Ab-B1-1704 did not inhibit HLA-G binding to LILRB2 expressed on THP1 cells, while anti-LILRB2 antibody Ab-B2-1825 inhibited HLA-G binding to LILRB2 expressed on THP1 cells.

FIG. 2 depicts that in Example 3, LILRB1×LILRB2 bispecific antibody BsAb-1882 binds to both huLILRB1 and huLILRB2 proteins, and that BsAb-1882 does not bind to huLILRB3, B4, B5 protein, or huLILRA1, A2, A3, A4 or A5 protein.

FIG. 3A depicts that in Example 4, LILRB1×LILRB2 bispecific antibodies BsAb-1882 and BsAb-1880 inhibited the binding of HLA-G to LILRB1 expressed on THP1 cells.

FIG. 3B depicts that in Example 4, LILRB1×LILRB2 bispecific antibodies BsAb-1882 showed anti-tumor efficacy in combination with anti-PD-1 antibody sasanlimab in a breast cancer xenograft.

FIG. 4 depicts that in Example 5, LILRB1×LILRB2 bispecific antibody BsAb-1882 showed anti-tumor efficacy when administered in combination with anti-PD-1 antibody sasanlimab in a breast cancer xenograft.

### DETAILED DESCRIPTION

The present invention may be understood more readily by reference to the following detailed description of the embodiments of the invention and the Examples included herein. It is to be understood that this invention is not limited to specific methods of making that may of course vary. It is to be also understood that the terminology used herein is for the purpose of describing specific embodiments only and is not intended to be limiting.

Exemplary embodiments (E) of the invention provided herein include:

E1. An isolated bispecific antibody comprising two binding arms, one arm binds to LILRB1 (B1 arm), and the other arm binds to LILRB2 (B2 arm), the B1 arm comprises a B1 arm heavy chain comprising a B1 arm heavy chain variable region (VH) and a B1 arm light chain comprising a B1 arm light chain variable region (VL), and the B2 arm comprises a B2 arm heavy chain comprising a B2 arm VH and a B2 arm light chain comprising a B2 arm VL, wherein
(i) the B1 arm VH comprises a VH CDR1 having the amino acid sequence shown in SEQ ID NO: 9, 10 or 11, a VH CDR2 having the amino acid sequence shown in SEQ ID NO: 12 or 13, and a VH CDR3 having the amino acid sequence shown in SEQ ID NO: 14;
(ii) the B1 arm VL comprises a VL CDR1 having the amino acid sequence shown SEQ ID NO: 2, a VL CDR2 having the amino acid sequence shown in SEQ ID NO: 3, and a VL CDR3 having the amino acid sequence shown in SEQ ID NO: 4;
(iii) the B2 arm VH comprises a VH CDR1 having the amino acid sequence shown in SEQ ID NO: 24, 25 or 26, a VH CDR2 having the amino acid sequence shown in SEQ ID NO: 27 or 28, and a VH CDR3 having the amino acid sequence shown in SEQ ID NO: 29; and
(iv) the B2 arm VL comprises a VL CDR1 having the amino acid sequence shown SEQ ID NO: 18, a VL CDR2 having the amino acid sequence shown in SEQ ID NO: 19, and a VL CDR3 having the amino acid sequence shown in SEQ ID NO: 20.

E1a. The isolated bispecific antibody of E1, wherein
(i) the B1 arm VH comprises a VH CDR1 having the amino acid sequence shown in SEQ ID NO: 9, a VH CDR2 having the amino acid sequence shown in SEQ ID NO: 12, and a VH CDR3 having the amino acid sequence shown in SEQ ID NO: 14, and the VH CDR1 and the VH CDR2 are by the Kabat definition;
(ii) the B1 arm VL comprises a VL CDR1 having the amino acid sequence shown SEQ ID NO: 2, a VL CDR2 having the amino acid sequence shown in SEQ ID NO: 3, and a VL CDR3 having the amino acid sequence shown in SEQ ID NO: 4;
(iii) the B2 arm VH comprises a VH CDR1 having the amino acid sequence shown in SEQ ID NO: 24, a VH CDR2 having the amino acid sequence shown in SEQ ID NO: 27, and a VH CDR3 having the amino acid sequence shown in SEQ ID NO: 29, and the VH CDR1 and the VH CDR2 are by the Kabat definition; and
(iv) the B2 arm VL comprises a VL CDR1 having the amino acid sequence shown SEQ ID NO: 18, a VL CDR2 having the amino acid sequence shown in SEQ ID NO: 19, and a VL CDR3 having the amino acid sequence shown in SEQ ID NO: 20.

E1b. The isolated bispecific antibody of E1, wherein
(i) the B1 arm VH comprises a VH CDR1 having the amino acid sequence shown in SEQ ID NO: 10, a VH CDR2 having the amino acid sequence shown in SEQ ID NO: 13, and a VH CDR3 having the amino acid sequence shown in SEQ ID NO: 14, and the VH CDR1 and the VH CDR2 are by the Chothia definition;
(ii) the B1 arm VL comprises a VL CDR1 having the amino acid sequence shown SEQ ID NO: 2, a VL CDR2 having the amino acid sequence shown in SEQ ID NO: 3, and a VL CDR3 having the amino acid sequence shown in SEQ ID NO: 4;
(iii) the B2 arm VH comprises a VH CDR1 having the amino acid sequence shown in SEQ ID NO: 25, a VH CDR2 having the amino acid sequence shown in SEQ ID NO: 28, and a VH CDR3 having the amino acid sequence shown in SEQ ID NO: 29, and the VH CDR1 and the VH CDR2 are by the Chothia definition; and
(iv) the B2 arm VL comprises a VL CDR1 having the amino acid sequence shown SEQ ID NO: 18, a VL CDR2 having the amino acid sequence shown in SEQ ID NO: 19, and a VL CDR3 having the amino acid sequence shown in SEQ ID NO: 20.

E1c. The isolated bispecific antibody of E1, wherein
(i) the B1 arm VH comprises a VH CDR1 having the amino acid sequence shown in SEQ ID NO: 11, a VH CDR2 having the amino acid sequence shown in SEQ ID NO: 12, and a VH CDR3 having the amino acid sequence shown in SEQ ID NO: 14, and the VH CDR1 is the extended VH CDR1, and the VH CDR2 is by the Kabat definition;
(ii) the B1 arm VL comprises a VL CDR1 having the amino acid sequence shown SEQ ID NO: 2, a VL CDR2 having the amino acid sequence shown in SEQ ID NO: 3, and a VL CDR3 having the amino acid sequence shown in SEQ ID NO: 4;
(iii) the B2 arm VH comprises a VH CDR1 having the amino acid sequence shown in SEQ ID NO: 26, a VH CDR2 having the amino acid sequence shown in SEQ ID NO: 27, and a VH CDR3 having the amino acid sequence shown in SEQ ID NO: 29, and the VH CDR1 is the extended VH CDR1, and the VH CDR2 is by the Kabat definition; and
(iv) the B2 arm VL comprises a VL CDR1 having the amino acid sequence shown SEQ ID NO: 18, a VL CDR2 having the amino acid sequence shown in SEQ ID NO: 19, and a VL CDR3 having the amino acid sequence shown in SEQ ID NO: 20.

E1d. The isolated bispecific antibody of E1, wherein
(i) the B1 arm VH comprises a VH CDR1 having the amino acid sequence shown in SEQ ID NO: 11, a VH CDR2 having the amino acid sequence shown in SEQ ID NO: 13, and a VH CDR3 having the amino acid sequence shown in SEQ ID NO: 14, and the VH CDR1 is the extended VH CDR1, and the VH CDR2 is by the Chothia definition;
(ii) the B1 arm VL comprises a VL CDR1 having the amino acid sequence shown SEQ ID NO: 2, a VL CDR2 having the amino acid sequence shown in SEQ ID NO: 3, and a VL CDR3 having the amino acid sequence shown in SEQ ID NO: 4;
(iii) the B2 arm VH comprises a VH CDR1 having the amino acid sequence shown in SEQ ID NO: 26, a VH CDR2 having the amino acid sequence shown in SEQ ID NO: 28, and a VH CDR3 having the amino acid sequence shown in SEQ ID NO: 29, and the VH CDR1 is the extended VH CDR1, and the VH CDR2 is by the Chothia definition; and
(iv) the B2 arm VL comprises a VL CDR1 having the amino acid sequence shown SEQ ID NO: 18, a VL CDR2 having the amino acid sequence shown in SEQ ID NO: 19, and a VL CDR3 having the amino acid sequence shown in SEQ ID NO: 20.

E2. An isolated bispecific antibody comprising two binding arms, one arm binds to LILRB1 (B1 arm), and the other arm binds to LILRB2 (B2 arm), the B1 arm comprises a B1 arm heavy chain comprising a B1 arm heavy chain variable region (VH) and a B1 arm light chain comprising a B1 arm light chain variable region (VL), and the B2 arm comprises a B2 arm heavy chain comprising a B2 arm VH and a B2 arm light chain comprising a B2 arm VL, wherein:
(i) the B1 arm VH comprises the amino acid sequence shown in SEQ ID NO: 16.
(ii) the B1 arm VL comprises the amino acid sequence shown in SEQ ID NO: 6,
(iii) the B2 arm VH comprises the amino acid sequence shown in SEQ ID NO: 31, and
(iv) the B2 arm VL comprises the amino acid sequence shown in SEQ ID NO: 22.

E2a. An isolated bispecific antibody comprising two binding arms, one arm binds to LILRB1 (B1 arm), and the other arm binds to LILRB2 (B2 arm), the B1 arm comprises a B1 arm heavy chain comprising a B1 arm heavy chain variable region (VH) and a B1 arm light chain comprising a B1 arm light chain variable region (VL), and the B2 arm comprises a B2 arm heavy chain comprising a B2 arm VH and a B2 arm light chain comprising a B2 arm VL, wherein:
(i) the B1 arm VH comprises the amino acid sequence encoded by the nucleic acid sequence of the insert of the plasmid deposited with ATCC having ATCC Accession No. PTA_127715;
(ii) the B1 arm VL comprises the amino acid sequence encoded by the nucleic acid sequence of the insert of the plasmid deposited with ATCC having ATCC Accession No. PTA_127716;
(iii) the B2 arm VH comprises the amino acid sequence encoded by the nucleic acid sequence of the insert of the plasmid deposited with ATCC having ATCC Accession No. PTA_127717, and
(iv) the B2 arm VL comprises the amino acid sequence encoded by the nucleic acid sequence of the insert of the plasmid deposited with ATCC having ATCC Accession No. PTA_127718.

E3. The isolated bispecific antibody of E1 E2, or E2a, wherein the bispecific antibody is a human IgG antibody.

E3a. The isolated bispecific antibody of E1, E2, E2a or E3, wherein:
(i) the B1 arm comprises a B1 arm light chain comprising, from its N terminus to C terminus the B1 arm VL, an IgG CL, a first IgG hinge or a fragment thereof and a first IgG Fc chain;
(ii) the B1 arm comprises a B1 arm heavy chain comprising, from its N terminus to C terminus, the B1 arm VH, an IgG CH1 and an optional fragment of an IgG hinge;
(iii) the B2 arm comprises a B2 arm light chain, comprising from its N terminus to C terminus the B2 arm VL and an IgG CL; and
(iv) the B2 arm comprises a B2 arm heavy chain, comprising from its N terminus to C terminus the B2 arm VH, an IgG CH1, a second IgG hinge or a fragment thereof, and the second IgG Fc chain.

E3b. The isolated bispecific antibody of E1 E2, or E2a, wherein the bispecific antibody is a human IgG antibody, and the B1 arm comprises a first IgG heavy chain constant region, and the B2 arm comprises a second IgG heavy chain constant region. Preferably, the bispecific antibody is a human IgG1 antibody where the B1 arm comprises a first human IgG1 heavy chain constant region, and the B2 arm comprises a second human IgG1 heavy chain constant region.

E4. The bispecific antibody of E3a or E3b, wherein the bispecific antibody is a full length human IgG1 antibody.

E4a. The isolated bispecific antibody of E1 E2 or E2a, wherein:
(v) the B1 arm comprises a B1 arm light chain comprising, from its N terminus to C terminus the B1 arm VL, an IgG1 CL, a first IgG1 hinge or a fragment thereof and a first IgG1 Fc chain;
(vi) the B1 arm comprises a B1 arm heavy chain comprising, from its N terminus to C terminus, the B1 arm VH, an IgG1 CH1 and an optional fragment of an IgG1 hinge;
(vii) the B2 arm comprises a B2 arm light chain comprising, from its N terminus to C terminus, the B2 arm VL and an IgG1 CL; and
(viii) the B2 arm comprises a B2 arm heavy chain, comprising from its N terminus to C terminus the B2 arm VH, an IgG1 CH1, a second IgG1 hinge region, and a second IgG1 Fc chain.

E4b. The isolated bispecific antibody of E3, E3a, E3b, E4 or E4a, wherein (a) the first IgG or IgG1 heavy chain constant region, or the first IgG or IgG1 Fc chain each comprises the mutations of 354C, 366S, 368A and/or 407V, and the second IgG or IgG1 heavy chain constant region, or the second IgG or IgG1 Fc chain each comprises the mutations of 366W and/or 349C; or (b) the second IgG or IgG1 heavy chain constant region, or the second IgG or IgG1 Fc chain each comprises the mutations of 354C, 366S, 368A and/or 407V, and the first IgG or IgG1 heavy chain constant region, or the first IgG or IgG1 Fc chain each comprises the mutations of 366W and/or 349C; all according to EU numbering.

E4c. The isolated bispecific antibody of E3, E3a, E3b, E4 or E4a, wherein (a) the first IgG or IgG1 heavy chain constant region, or the first IgG or IgG1 hinge region or the fragment thereof and the first IgG or IgG1 Fc chain, each comprises the mutations of 221R and/or 409R; and the second IgG or IgG1 heavy chain constant region, or the second IgG or IgG1 hinge or a fragment thereof and the second IgG or IgG1 Fc chain, each comprises the mutations of 221E and/or 368E; or (b) the second IgG or IgG1 heavy chain constant region, or the second IgG or IgG1 hinge region or the fragment thereof and the second IgG or IgG1 Fc chain, each comprises the mutations of 221R and/or 409R, and the first IgG or IgG1 heavy chain constant region, or the first IgG or IgG1 hinge or a fragment thereof and the first IgG or IgG1 Fc chain, each comprises the mutations of 221E and/or 368E; all according to EU numbering.

E4d. The isolated bispecific antibody of E4b or E4c, wherein each of the first and second IgG or IgG1 heavy chain constant region or each of the first and second IgG or IgG1 Fc chain further comprises the mutations of 234A, 235A and 237A, all according to EU numbering.

E5. An isolated bispecific antibody comprising two binding arms, one arm binds to LILRB1 (B1 arm), and the other arm binds to LILRB2 (B2 arm), the B1 arm comprises a B1 arm heavy chain and a B1 arm light chain, and the B2 arm comprises a B2 arm heavy chain and a B2 arm light chain, wherein:
(i) the B1 arm heavy chain comprises the amino acid sequence shown in SEQ ID No 8, and the B1 arm light chain comprises the amino acid sequence shown in SEQ ID NO: 1 or SEQ ID NO: 35; and
(ii) the B2 arm heavy chain comprises the amino acid sequence shown in SEQ ID No: 23 or SEQ ID NO: 36, and the B2 arm light chain comprises the amino acid sequence shown in SEQ ID NO: 17.

E6. A pharmaceutical composition comprising the bispecific antibody any one of E1, E1 a-d, E2, E2a, E3, E3a, E4, E4a and E5.

E7. A method to treat cancer in a subject comprising administering to the subject the bispecific antibody of any one of E1, E1 a-d, E2, E2a E3, E3a, E4, E4a and E5 or the pharmaceutical composition of E6.

E7a. The method of E7, further comprising administering to the subject a PD-1 antibody. In some embodiments, the PD-1 antibody is sasanlimab. In some embodiments, the cancer is breast cancer.

E7b. The method of E7a, wherein the administration of the bispecific antibody enhances the anti-tumor efficacy of the PD-1 antibody.

E8. A polynucleotide encoding at least one of the (i) B1 arm VH, (ii) B1 arm VL, (iii) B2 arm VH, and (iv) B2 arm VL, of the bispecific antibody of any one of E1, E1 a-d, E2, E2a, E3, E3a, E4, E4a and E5.

E9. A polynucleotide encoding at least one of the (i) B1 arm heavy chain, (ii) B1 arm light chain, (iii) B2 arm heavy chain, and (iv) B2 arm light chain, of the bispecific antibody of any one of E1, E1 a-d, E2, E2a, E3, E3a, E4, E4a and E5.

E10. A vector comprising the polynucleotide of E8 or E9, or both the polynucleotide of E8 and the polynucleotide of E9.

E11. A host cell comprising the vector of E10.

E12. An isolated anti-LILRB1 antibody comprising a heavy chain variable region (VH) and a light chain variable region (VL), wherein:
(i) VH comprises a VH CDR1 having the amino acid sequence shown in SEQ ID NO: 9, 10 or 11, a VH CDR2 having the amino acid sequence shown in SEQ ID NO: 12 or 13, and a VH CDR3 having the amino acid sequence shown in SEQ ID NO: 14; and
(ii) the VL comprises a VL CDR1 having the amino acid sequence shown SEQ ID NO: 2, a VL CDR2 having the amino acid sequence shown in SEQ ID NO: 3, and a VL CDR3 having the amino acid sequence shown in SEQ ID NO: 4.

E13. An isolated anti-LILRB1 antibody comprising a VH that comprises the amino acid sequence shown in SEQ ID NO: 16, and a VL that comprises amino acid sequence shown in SEQ ID NO: 6.

E13a. An isolated anti-LILRB1 antibody comprising a VH that comprises the amino acid sequence encoded by the nucleic acid sequence of the insert of the plasmid deposited with ATCC having ATCC Accession No. PTA_127715, and a VL that comprises the amino acid sequence encoded by the nucleic acid sequence of the insert of the plasmid deposited with ATCC having ATCC Accession No. PTA_127716.

E14. The isolated anti-LILRB1 antibody of E12 E13 or E13a, wherein the antibody is a full length human IgG antibody, with two identical heavy chains and two identical light chains.

E15. The isolated anti-LILRB1 antibody of E14, wherein the antibody is a full length human IgG1 antibody.

E16. An isolated anti-LILRB2 antibody comprising a heavy chain variable region (VH) and a light chain variable region (VL), wherein:
(i) VH comprises a VH CDR1 having the amino acid sequence shown in SEQ ID NO: 24, 25 or 26, a VH CDR2 having the amino acid sequence shown in SEQ ID NO: 27 or 28, and a VH CDR3 having the amino acid sequence shown in SEQ ID NO: 29; and
(ii) the VL comprises a VL CDR1 having the amino acid sequence shown SEQ ID NO: 18, a VL CDR2 having the amino acid sequence shown in SEQ ID No: 19, and a VL CDR3 having the amino acid sequence shown in SEQ ID No: 20.

E17. An isolated anti-LILRB2 antibody comprising a VH that comprises the amino acid sequence shown in SEQ ID NO: 31, and a VL that comprises amino acid sequence shown in SEQ ID NO: 22.

E17a. An isolated anti-LILRB2 antibody comprising a VH that comprises the amino acid sequence encoded by the nucleic acid sequence of the insert of the plasmid deposited with ATCC having ATCC Accession No. PTA_127717, and a VL that comprises the amino acid sequence encoded by the nucleic acid sequence of the insert of the plasmid deposited with ATCC having ATCC Accession No. PTA_127718

E18. The isolated anti-LILRB2 antibody of E16, E17 or E17a, wherein the antibody is a full length human IgG antibody, with two identical heavy chains and two identical light chains.

E19. The isolated anti-LILRB2 antibody of E18, wherein the antibody is a full length human IgG1 antibody.

E20. A pharmaceutical composition comprising the anti-LILRB1 antibody of any one of E12, E13, E13a, E14 and E15, or the anti-LILRB2 antibody of any one of E16, E17, E17a, E18 and E19.

E21. A method to treat cancer in a subject comprising administering to the subject the LILRB1 antibody of any one of E12 E13, E13a, E14 and E15, the anti-LILR2 antibody of any one of E16 E17, E17a, E18 and E19, or the pharmaceutical composition of E20.

E22. An isolated polynucleotide encoding
(i) the VH, the VL or both the VH and VL of the anti-LILRB1 antibody of any one of E12 E13, E13a, E14 and E15; or
(ii) the VH, the VL or both the VH and VL of the anti-LILRB2 antibody of any one of claims E16 E17, E17a, E18 and E19.

E23. A vector comprising at least one polynucleotide of E22.

E24. A host cell comprising the vector of E23.

E25. The isolated bispecific antibody of any one of E1, E1 a-d, E2, E2a E3, E3a, E4, E4a and E5, wherein the bispecific antibody does not bind to huLILRA1, huLILRA2, huLILRA3, huLILRA4, huLILRA5, huLILRA6, huLILRB3, huLILRB4 and/or huLILRB5.

E26. The isolated bispecific antibody of any one of E1, E1 a-d, E2, E2a E3, E3a, E4, E4a, E5 and E25, wherein the bispecific antibody binds to huLILRB1 with a binding affinity (K_{D}) of about 30 nM, about 5-150 nM or about 5-1000 nM, and binds to huLILRB2 with a binding affinity of about 0.7 nM, about 0.1-10 nM or about 0.1-100 nM.

E27. The isolated bispecific antibody of E25 or E26, wherein the binding affinity is measured by surface plasmon resonance.

E28. The isolated bispecific antibody of any one of E1, E1 a-d, E2, E2a E3, E3a, E4, E4a, E5, and E25-E27, wherein the bispecific antibody inhibits the interactions between HLA-G and HuLILRB1, and the interactions between HLA-G and LILRB2.

E29. The isolated bispecific antibody of E28, wherein the bispecific antibody inhibits the interactions between HLA-G and HuLILRB1 with an IC50 of about 40 nM, about 10-200 nM, or about 5-1000 nM, and inhibits the interactions between HLA-G and LILRB2 with an IC50 of about 15 nM, about 5-100 nM or about 5-1000 nM.

E30. The isolated bispecific antibody of E29, wherein the inhibition is measured by a cell based ligand competition assay for the inhibition of the interaction of recombinant HLA-G tetramer with either LILRB1 or LILRB2 expressed on THP1 cells.

E30a. The isolated bispecific antibody of any one of E1, E1 a-d, E2, E2a E3, E3a, E4, E4a and E5, wherein the bispecific antibody has anti-tumor efficacy in combination with an anti-PD-1 antibody in treating cancer. In some embodiments, the anti-PD-1 antibody is sasanlimab. In some embodiments, the cancer is breast cancer.

E30b. The isolated bispecific antibody of any one of E1, E1 a-d, E2, E2a E3, E3a, E4, E4a and E5, wherein the bispecific antibody reverses HLA-G mediated inhibition of TNF-α production in primary monocytes and macrophages. In some embodiments, the bispecific antibody reverses HLA-G inhibition of TNF-α release with average IC50 values of about 1.2 nM, about 0.1 to 4.0 nM, or about 0.1 to 10 nM for monocytes and about 28.0 nM, about 10 to 40 nM, or about 10 to 100 nM for macrophages.

E31. The isolated anti-LILRB1 antibody of any one of E12, E13, E13a, E14 and E15, wherein the antibody binds to huLILRB1 and does not bind to huLILRA1, huLILRA2 and/or huLILRA5.

E32. The isolated anti-LILRB1 antibody of E31, wherein the antibody binds to huLILRB1 with a binding affinity (K_{D}) of about 26 nM, about 10 nM to 100 nM, or about 5 nM to 1000 nM.

E33. The isolated anti-LILRB1 antibody of E31 or E32, wherein the binding affinity (K_{D}) is measured by surface plasmon resonance.

E34. The isolated anti-LILRB1 antibody of any one of E12, E13, E13a, E14 and E15, wherein the antibody inhibits the interaction between HLA-G tetramer with huLILRB1, with an EC50 of about 3 nM, about 1-10 nM or about 1-100 nM, but does not inhibit the interaction between HLA-G tetramer with huLILRB2, in an cell based ligand competition assay for the inhibition of the interaction of recombinant HLA-G tetramer with either LILRB1 or LILRB2 expressed on THP1 cells.

E35. The isolated anti-LILRB2 antibody of any one of E16, E17, E17a, E18 and E19, wherein the antibody binds to huLILRB2 and does not bind to huLILRA1, huLILRA2 and/or huLILRA5.

E36. The isolated anti-LILRB2 antibody of E35, wherein the antibody binds to huLILRB2 with a binding affinity (K_{D}) of about 0.6 nM, about 0.1 nM to 10 nM or about 0.1 nM-100 nM.

E37. The isolated anti-LILRB2 antibody of E35 or E36, wherein the binding affinity (K_{D}) is measured by surface plasmon resonance.

E38. The isolated anti-LILRB2 antibody of any one of E16, E17, E17a, E18 and E19, wherein the antibody inhibits the interaction between HLA-G tetramer with huLILRB2, with an EC50 of about 3 nM, about 1-10 nM or about 1-100 nM, but does not inhibit the interaction between HLA-G tetramer with huLILRB1, in an cell based ligand competition assay for the inhibition of the interaction of recombinant HLA-G tetramer with either LILRB1 or LILRB2 expressed on THP1 cells.

E39. A method of producing the bispecific antibody of E1, E1a-d, E2, E2a, E3, E3a-b, E4, E4a-4d or E5, the LILRB1 antibody of E12, E13, E13a, E14 or E15, and the LILRB2 antibody of E16, E17, E17a, E18 or E19, comprising culturing the host cell of E12 and E24, respectively, under conditions that result in production of the bispecific antibody, the LILRB1 antibody and the LILRB2 antibody, respectively, and recovering the bispecific antibody, the LILRB1 antibody and the LILRB2 antibody, respectively.

The section headings used herein are for organizational purposes only and are not to be construed as limiting the subject matter described.

All references cited herein, including patent applications, patent publications, UniProtKB accession numbers are herein incorporated by reference, as if each individual reference were specifically and individually indicated to be incorporated by reference in its entirety.

The techniques and procedures described or referenced herein are generally well understood and commonly employed using conventional methodology by those skilled in the art, such as, for example, the widely utilized methodologies described in Sambrook et al, Molecular Cloning: A Laboratory Manual 3rd. edition (2001) Cold Spring Harbor Laboratory Press, Cold Spring Harbor, N.Y. CURRENT PROTOCOLS IN MOLECULAR BIOLOGY (F. M. Ausubel, et al. eds., (2003)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds); Gene Transfer Vectors for Mammalian Cells (J. M. Miller and M. P. Calos, eds., 1987); PCR: The Polymerase Chain Reaction, (Mullis et al, eds., 1994); Current Protocols in Immunology (J. E. Coligan et al, eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999)); The Antibodies (M. Zanetti and J. D. Capra, eds., Harwood Academic Publishers, 1995); and updated versions thereof.

### Definitions

Unless otherwise defined herein, scientific and technical terms used in connection with the present invention have the meanings that are commonly understood by those of ordinary skill in the art.

As used herein, the singular form “a”, “an”, and “the” include plural references unless indicated otherwise. For example, “an” antibody includes one or more antibodies.

Where aspects or embodiments of the invention are described in terms of a Markush group or other grouping of alternatives, the present invention encompasses not only the entire group listed as a whole, but each member of the group individually and all possible subgroups of the main group, but also the main group absent one or more of the group members. The present invention also envisages the explicit exclusion of one or more of any of the group members in the claimed invention.

Any example(s) following the term “e.g.” or “for example” is not meant to be exhaustive or limiting.

As used herein, the term “about” when used to modify a numerically defined parameter (e.g., the dose of ***) means that the parameter may vary by as much as 10% below or above the stated numerical value for that parameter. For example, a dose of about 5 mg means 5%±10%, i.e. it may vary between 4.5 mg and 5.5 mg.

An “antibody” refers to an immunoglobulin molecule capable of specific binding to a target, such as a polypeptide, carbohydrate, polynucleotide, lipid, etc., through at least one antigen binding site, located in the variable region of the immunoglobulin molecule.

As used herein, the term “antibody” can encompass any type of antibody (e.g. monospecific, bispecific), and includes portions of intact antibodies that retain the ability to bind to a given antigen (e.g. an “antigen-binding fragment”), and any other modified configuration of an immunoglobulin molecule that comprises an antigen binding site.

An antibody includes an antibody of any class, such as IgG, IgA, or IgM (or sub-class thereof), and the antibody need not be of any particular class. Depending on the antibody amino acid sequence of the constant region of its heavy chains (HC), immunoglobulins can be assigned to different classes. There are five major classes of immunoglobulins: IgA, IgD, IgE, IgG, and IgM, and several of these may be further divided into subclasses (isotypes), e.g., IgG₁, IgG₂, IgG₃, IgG₄, IgA₁ and IgA₂. The heavy chain constant regions that correspond to the different classes of immunoglobulins are called alpha, delta, epsilon, gamma, and mu, respectively. The subunit structures and three-dimensional configurations of different classes of immunoglobulins are well known.

A human or humanized “IgG1”, “IgG2”, “IgG3” and “IgG4” antibody “, refers to a human or humanized antibody as defined herein, and that
(i) has a first and a second heavy chains same or different and comprising a first VH and a second VH respectively, has a first and second light chains same or different comprising a first VL and a second VL respectively, the first VH and the first VL form a first binding domain and the second VH and second VL form a second binding domain, and the first binding domain and the second binding domain bind to the same target or two different targets, and
(ii) (a) each heavy chain has a constant region that has an amino acid sequence that is at least 95% identical to (a) a human wildtype IgG1 heavy chain constant region having the amino acid sequence shown in SEQ ID NO: 43; (b) a human wildtype IgG2 heavy chain constant region having the amino acid sequence shown in SEQ ID NO: 44; (c) a human wildtype IgG3 heavy chain constant region having the amino acid sequence shown in SEQ ID NO: 45; and (d) a human wildtype IgG4 heavy chain constant region having the amino acid sequence shown in SEQ ID NO: 46, respectively; or
(ii) (b) the antibody comprises two human IgG1, two human IgG2, two human IgG3 or two human IgG4 Fc chains (a first and a second) respectively, and each of the first and second Fc chain is fused at its N terminus to the C terminus of a first and a second human IgG1, IgG2, IgG3, and IgG4 hinge region, or a fragment thereof, respectively, and the N terminus of the first and the second hinge region is each fused to the C terminus of a first and a second human IgG1, IgG2, IgG3 and IgG4 CH1 region, respectively, or a human IgG light chain constant (CL) region, and the first and second CH or CL is fused at its N terminus to the C terminus of the first VH or first VL and the second VH or second VL, respectively.

In some embodiments of a human or humanized IgG1 antibody as defined above, the antibody is BsAb-1882, BsAb-1880, Ab-B1-1704 or Ab-B2-1825, as described in Tables 1, 2 and 3.

A human or humanized “IgG antibody” refers to an antibody that is a human or humanized IgG1, IgG2, IgG3 or IgG4 antibody.

A human IgG light chain constant region (CL) refers to a light chain constant region having an amino acid sequence that is at least 95% identical to a human wildtype IgG light chain constant region having the amino acid sequence shown in SEQ ID NO: 47, 48 or 49.

A human IgG1, IgG2, IgG3 and IgG4 heavy chain constant region, the corresponding CH1 region (“CH1”), hinge region (“hinge”), CH2 region (“CH2”) and CH3 region (“CH3”), Fc chain and other structural components thereof are similarly defined as having an amino acid sequence at least 95% identical to the amino acid sequence of a human wildtype IgG1, IgG2, IgG3 and IgG4 heavy chain constant region having the amino acid sequence of SEQ ID NO: 43, 44, 45 and 46, respectively, or the corresponding portions thereof as defined under the International Immunogenetics Information System® (IMGT) or as defined herein. See also Edelman, G. M. et al., Proc. Natl. Acad. USA, 63, 78-85 (1969) and Kabat, E. A. et al., Sequences of proteins of immunological interest. 5th Edition-US Department of Health and Human Services, NIH publication n° 91-3242, U.S. Plant Pat. No. 662,680,689 (1991).

A fragment of a human IgG1, IgG2, IgG3 and IgG4 hinge region (or hinge) refers to an amino acid sequence that is a portion of the human IgG1, IgG2, IgG3 and IgG4 hinge region, respectively, and comprises at least three amino acid residues, and preferably comprises 4, 5, 6, 7, 8, 9, 10, 11, 12, 13 or 14 amino acid residues of the human IgG1 hinge region, 4, 5, 6, 7, 8, 9, 10, or 11 amino acid residues of the human IgG2 hinge region, 4, 5, 6, 7, 8, 9, 10, 11, 12, 13, 14 . . . and up to 61 amino acid resides of the human IgG3 hinge region, and 4, 5, 6, 7, 8, 9, 10, or 11 amino acid residues of the human IgG4 hinge region, respectively.

A “full length human or humanized IgG1 antibody” refers to a human or humanized IgG1 antibody as described above, that comprises a first and second IgG1 CH1, and a first and a second human IgG1 CL. A “full length human or humanized IgG2, IgG3 or IgG4 antibody” is similarly defined. A full length human or humanized IgG antibody is a full length human or humanized IgG1, IgG2, IgG3 or IgG4 antibody.

Examples of antibody antigen-binding fragments and modified configurations include (i) a Fab fragment (a monovalent fragment consisting of the VL, VH, CL and CH1 domains); (ii) a F(ab′)₂ fragment (a bivalent fragment comprising two Fab fragments linked by a disulfide bridge at the hinge region); and (iii) a Fv fragment consisting of the VL and VH domains of a single arm of an antibody. Furthermore, although the two domains of an Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv)); see e.g., Bird et al., Science 1988; 242:423-426 and Huston et al., Proc. Natl. Acad. Sci. 1988 USA 85:5879-5883. Other forms of single chain antibodies, such as diabodies are also encompassed.

In addition, further encompassed are antibodies that are missing a C-terminal lysine (K) amino acid residue on a heavy chain polypeptide (e.g. human IgG1 heavy chain comprises a terminal lysine). As is known in the art, the C-terminal lysine is sometimes clipped during antibody production, resulting in an antibody with a heavy chain lacking the C-terminal lysine. Alternatively, an antibody heavy chain may be produced using a nucleic acid that does not include a C-terminal lysine.

A “variable region” of an antibody refers to the variable region of the antibody light chain or the variable region of the antibody heavy chain, either alone or in combination. As known in the art, the variable regions of the heavy and light chains each consist of four framework regions (FRs) connected by three complementarity determining regions (CDRs) also known as hypervariable regions, and contribute to the formation of the antigen binding site of antibodies. If variants of a subject variable region are desired, particularly with substitution in amino acid residues outside of a CDR region (i.e., in the framework region), appropriate amino acid substitution, preferably, conservative amino acid substitution, can be identified by comparing the subject variable region to the variable regions of other antibodies which contain CDR1 and CDR2 sequences in the same canonincal class as the subject variable region (Chothia and Lesk, J Mol Biol 196 (4): 901-917, 1987).

In certain embodiments, definitive delineation of a CDR and identification of residues comprising the binding site of an antibody is accomplished by solving the structure of the antibody or solving the structure of the antibody-ligand complex. In certain embodiments, that can be accomplished by any of a variety of techniques known to those skilled in the art, such as X-ray crystallography. In certain embodiments, various methods of analysis can be employed to identify or approximate the CDR regions. In certain embodiments, various methods of analysis can be employed to identify or approximate the CDR regions. Examples of such methods include, but are not limited to, the Kabat definition, the Chothia definition, the AbM definition, the contact definition, the extended definition, and the conformational definition.

The Kabat definition is a standard for numbering the residues in an antibody and is typically used to identify CDR regions. See, e.g., Johnson & Wu, 2000, Nucleic Acids Res., 28:214-8. The Chothia definition is similar to the Kabat definition, but the Chothia definition takes into account positions of certain structural loop regions. See, e.g., Chothia et al., 1986, J. Mol. Biol., 196:901-17; Chothia et al., 1989, Nature, 342:877-83. The extended definition is the combination of the Kabat and Chothia definitions. The AbM definition uses an integrated suite of computer programs produced by Oxford Molecular Group that model antibody structure. See, e.g., Martin et al., 1989, Proc Natl Acad Sci (USA), 86:9268-9272; “AbM™, A Computer Program for Modeling Variable Regions of Antibodies,” Oxford, UK; Oxford Molecular, Ltd. The AbM definition models the tertiary structure of an antibody from primary sequence using a combination of knowledge databases and ab initio methods, such as those described by Samudrala et al., 1999, “Ab Initio Protein Structure Prediction Using a Combined Hierarchical Approach,” in PROTEINS, Structure, Function and Genetics Suppl., 3:194-198. The contact definition is based on an analysis of the available complex crystal structures. See, e.g., MacCallum et al., 1996, J. Mol. Biol., 5:732-45. In another approach, referred to herein as the “conformational definition” of CDRs, the positions of the CDRs may be identified as the residues that make enthalpic contributions to antigen binding. See, e.g., Makabe et al., 2008, Journal of Biological Chemistry, 283:1156-1166. Still other CDR boundary definitions may not strictly follow one of the above approaches, but will nonetheless overlap with at least a portion of the Kabat CDRs, although they may be shortened or lengthened in light of prediction or experimental findings that particular residues or groups of residues do not significantly impact antigen binding. As used herein, a CDR may refer to CDRs defined by any approach known in the art, including combinations of approaches. The methods used herein may utilize CDRs defined according to any of these approaches. For any given embodiment containing more than one CDR, the CDRs may be defined in accordance with any one or more of Kabat, Chothia, extended, AbM, contact, or conformational definitions.

A “constant region” of an antibody refers to the constant region of the antibody light chain or the constant region of the antibody heavy chain, either alone or in combination. An IgG heavy chain constant region contains three sequential immunoglobulin domains (CH1, CH2, and CH3), with a hinge region between the CH1 and CH2 domains. An IgG light chain constant region contains a single immunoglobulin domain (CL)

A “Fc domain” refers to the portion of an immunoglobulin (Ig) molecule that correlates to a crystallizable fragment obtained by papain digestion of an Ig molecule. As used herein, the term relates to the 2-chained constant region of an antibody, each chain excluding the first constant region immunoglobulin domain. Within an Fc domain, there are two “Fc chains” (e.g. a “first Fc chain” and a “second Fc chain”). “Fc chain” generally refers to the C-terminal portion of an antibody heavy chain. Thus, Fc chain refers to the last two constant region immunoglobulin domains (CH2 and CH3) of IgA, IgD, and IgG heavy chains, and the last three constant region immunoglobulin domains of IgE and IgM heavy chains, and optionally the flexible hinge N-terminal to these domains.

Although the boundaries of the Fc chain may vary, the human IgG heavy chain Fc chain is usually defined to comprise residues C226 or P230 to its carboxyl-terminus, wherein the numbering is according to the EU index of Edelman et al., Proc. Natl. Acad. Sci. USA 1969; 63 (1): 78-85 and as described in Kabat et al., 1991. Typically, the Fc chain comprises from about amino acid residue 236 to about 447 of the human IgG1 heavy chain constant region. “Fc chain” may refer to this polypeptide in isolation, or in the context of a larger molecule (e.g. in an antibody heavy chain or Fc fusion protein).

A “functional” Fc domain refers to an Fc domain that possesses at least one effector function of a native sequence Fc domain. Exemplary “effector functions” include C1q binding; complement dependent cytotoxicity (CDC); Fc receptor binding; antibody-dependent cell-mediated cytotoxicity (ADCC); phagocytosis; down-regulation of cell surface receptors (e.g., B cell receptor); and B cell activation, etc. Such effector functions generally require the Fc domain to be combined with a binding domain (e.g., an antibody variable region) and can be assessed using various assays known in the art for evaluating such antibody effector functions.

A “native sequence” Fc chain refers to a Fc chain that comprises an amino acid sequence identical to the amino acid sequence of an Fc chain found in nature. A “variant” Fc chain comprises an amino acid sequence which differs from that of a native sequence Fc chain by virtue of at least one amino acid modification

A “monoclonal antibody” (mAb) refers to an antibody that is derived from a single copy or clone, including e.g., any eukaryotic, prokaryotic, or phage clone. Monoclonal antibodies are highly specific, being directed against a single antigenic site. Furthermore, in contrast to polyclonal antibody preparations, which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler and Milstein, 1975, Nature 256:495, or may be made by recombinant DNA methods such as described in U.S. Pat. No. 4,816,567. In another example, monoclonal antibodies may be isolated from phage libraries such as those generated using the techniques described in McCafferty et al., 1990, Nature 348:552-554.

A “human antibody” refers to an antibody which possesses an amino acid sequence which corresponds to that of an antibody produced by a human or has been made using any technique for making fully human antibodies. For example, fully human antibodies may be obtained by using commercially available mice that have been engineered to express specific human immunoglobulin proteins, or by library (e.g. phage, yeast, or ribosome) display techniques for preparing fully human antibodies. This definition of a human antibody specifically excludes a humanized antibody comprising non-human antigen binding residues.

A “chimeric antibody” refers to an antibody in which the variable region sequences are derived from one species and the constant region sequences are derived from another species, such as an antibody in which the variable region sequences are derived from a mouse antibody and the constant region sequences are derived from a human antibody.

A “humanized” antibody refers to a non-human (e.g. murine) antibody that is a chimeric antibody that contains minimal sequence derived from non-human immunoglobulin. Preferably, humanized antibodies are human immunoglobulins (recipient antibody) in which residues from a CDR of the recipient are replaced by residues from a CDR of a non-human species (donor antibody) such as mouse, rat, or rabbit having the desired specificity, affinity, and capacity. The humanized antibody may comprise residues that are found neither in the recipient antibody nor in the imported CDR or framework sequences, but are included to further refine and optimize antibody performance.

An “antigen” refers to the molecular entity used for immunization of an immunocompetent vertebrate to produce the antibody that recognizes the antigen or to screen an expression library (e.g., phage, yeast or ribosome display library, among others) for antibody selection. Herein, antigen is termed more broadly and is generally intended to include target molecules that are specifically recognized by the antibody, thus including fragments or mimics of the molecule used in an immunization process for raising the antibody or in library screening for selecting the antibody.

An “epitope” refers to the area or region of an antigen to which an antibody specifically binds, e.g., an area or region comprising residues that interact with the antibody, as determined by any method well known in the art. There are many methods known in the art for mapping and characterizing the location of epitopes on proteins, including solving the crystal structure of an antibody-antigen complex, competition assays, gene fragment expression assays, epitope mapping, and synthetic peptide-based assays, as described, for example, in Chapter 11 of Harlow and Lane, Using Antibodies, a Laboratory Manual, Cold Spring Harbor Laboratory Press, Cold Spring Harbor, New York, 1999. In addition or alternatively, during the discovery process, the generation and characterization of antibodies may elucidate information about desirable epitopes. From this information, it is then possible to competitively screen antibodies for binding to the same epitope.

In addition, the epitope to which an antibody binds can be determined in a systematic screening by using overlapping peptides derived from the antigen and determining binding by the antibody. According to the gene fragment expression assays, the open reading frame encoding the antigen can be fragmented either randomly or by specific genetic constructions and the reactivity of the expressed fragments of the antigen with the antibody to be tested is determined. The gene fragments may, for example, be produced by PCR and then transcribed and translated into protein in vitro, in the presence of radioactive amino acids. The binding of the antibody to the radioactively labeled antigen fragments is then determined by immunoprecipitation and gel electrophoresis.

Certain epitopes can also be identified by using large libraries of random peptide sequences displayed on the surface of phage particles (phage libraries) or yeast (yeast display). Alternatively, a defined library of overlapping peptide fragments can be tested for binding to the test antibody in simple binding assays. In an additional example, mutagenesis of an antigen, domain swapping experiments and alanine scanning mutagenesis can be performed to identify residues required, sufficient, or necessary for epitope binding.

At its most detailed level, the epitope for the interaction between the antigen and the antibody can be defined by the spatial coordinates defining the atomic contacts present in the antigen-antibody interaction, as well as information about their relative contributions to the binding thermodynamics. At a less detailed level, the epitope can be characterized by the spatial coordinates defining the atomic contacts between the antigen and antibody. At a further less detailed level the epitope can be characterized by the amino acid residues that it comprises as defined by a specific criterion, e.g., by distance between atoms (e.g., heavy, i.e., non-hydrogen atoms) in the antibody and the antigen. At a further less detailed level the epitope can be characterized through function, e.g., by competition binding with other antibodies. The epitope can also be defined more generically as comprising amino acid residues for which substitution by another amino acid will alter the characteristics of the interaction between the antibody and antigen (e.g. using alanine scanning).

From the fact that descriptions and definitions of epitopes, dependent on the epitope mapping method used, are obtained at different levels of detail, it follows that comparison of epitopes for different antibodies on the same antigen can similarly be conducted at different levels of detail.

Epitopes described at the amino acid level, e.g., determined from an X-ray crystallography, Nuclear Magnetic Resonance (NMR) spectroscopy, hydrogen/deuterium exchange Mass Spectrometry (H/D-MS), are said to be identical if they contain the same set of amino acid residues. Epitopes are said to overlap if at least one amino acid is shared by the epitopes. Epitopes are said to be separate (unique) if no amino acid residue is shared by the epitopes.

Yet another method which can be used to characterize an antibody is to use competition assays with other antibodies known to bind to the same antigen, to determine if an antibody of interest binds to the same epitope as other antibodies. Competition assays are well known to those of skill in the art. Epitopes characterized by competition binding are said to be overlapping if the binding of the corresponding antibodies are mutually exclusive, i.e., binding of one antibody excludes simultaneous or consecutive binding of the other antibody. The epitopes are said to be separate (unique) if the antigen is able to accommodate binding of both corresponding antibodies simultaneously.

Epitopes can be linear or conformational. In a linear epitope, all of the points of interaction between the protein and the interacting molecule (such as an antibody) occur linearly along the primary amino acid sequence of the protein. A “nonlinear epitope” or “conformational epitope” comprises noncontiguous polypeptides (or amino acids) within the antigenic protein to which an antibody specific to the epitope binds.

The term “binding affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured by common methods known in the art. Low-affinity antibodies generally bind antigen slowly and tend to dissociate readily, whereas high-affinity antibodies generally bind antigen faster and tend to remain bound longer. In particular, the term “binding affinity” is intended to refer to the dissociation rate of a particular antigen-antibody interaction. The K_{D} is the ratio of the rate of dissociation, also called the “off-rate (K_{off})” or “k_{d}” to the association rate, or “on-rate (kₒₙ)” or “k_{d}”. Thus, K_{D} equals K_{off}/kₒₙ (or k_{d}/k_{d}) and is expressed as a molar concentration (M). It follows that the smaller the K_{D}, the stronger the affinity of binding. Therefore, a K_{D} of 1 μM indicates weaker binding affinity compared to a K_{D} of 1 nM. K_{D} values for antibodies can be determined using methods well established in the art. One exemplary method for determining the K_{D} of an antibody is by using surface plasmon resonance (SPR), typically using a biosensor system such as BIACORE system. BIACORE kinetic analysis comprises analyzing the binding and dissociation of an antigen from chips with immobilized molecules (e.g., molecules comprising epitope binding domains), on their surface. Another method for determining the K_{D} of an antibody is by using Bio-Layer Interferometry, typically using OCTET® technology (Octet QK^{e} system, ForteBio). Alternatively, or in addition, a KinExA (Kinetic Exclusion Assay) assay, available from Sapidyne Instruments (Boise, ID) can also be used.

A “monospecific antibody” refers to an antibody that comprises one or more antigen binding sites per molecule such that any and all binding sites of the antibody specifically recognize the identical epitope on the antigen. Thus, in cases where a monospecific antibody has more than one antigen binding site, the binding sites compete with each other for binding to one antigen molecule.

A “bispecific antibody” refers to a molecule that has binding specificity for at least two different epitopes. In some embodiments, bispecific antibodies can bind simultaneously two different antigens. In other embodiments, the two different epitopes may reside on the same antigen.

The term “half maximal effective concentration (EC50)” refers to the concentration of a therapeutic agent which causes a response halfway between the baseline and maximum after a specified exposure time. The therapeutic agent may cause inhibition or stimulation. The EC50 value is commonly used, and is used herein, as a measure of potency.

An “agonist” refers to a substance which promotes (i.e., induces, causes, enhances, or increases) the biological activity or effect of another molecule. The term agonist encompasses substances (such as an antibody) which bind to a molecule to promote the activity of that molecule.

An “antagonist” refers to a substance that prevents, blocks, inhibits, neutralizes, or reduces a biological activity or effect of another molecule, such as a receptor. The term antagonist encompasses substances (such as an antibody) which bind to a molecule to prevent or reduce the activity of that molecule.

The term “compete”, as used herein with regard to an antibody, means that a first antibody binds to an epitope in a manner sufficiently similar to the binding of a second antibody such that the result of binding of the second antibody with its cognate epitope is detectably decreased in the presence of the first antibody compared to the binding of the second antibody in the absence of the first antibody. The alternative, where the binding of the first antibody to its epitope is also detectably decreased in the presence of the second antibody, can, but need not be the case. That is, a first antibody can inhibit the binding of a second antibody to its epitope without that second antibody inhibiting the binding of the first antibody to its respective epitope. However, where each antibody detectably inhibits the binding of the other antibody with its cognate epitope or ligand, whether to the same, greater, or lesser extent, the antibodies are said to “cross-compete” with each other for binding of their respective epitope(s). Both competing and cross-competing antibodies are encompassed by the present invention. Regardless of the mechanism by which such competition or cross-competition occurs (e.g., steric hindrance, conformational change, or binding to a common epitope, or portion thereof), the skilled artisan would appreciate, based upon the teachings provided herein, that such competing or cross-competing antibodies are encompassed and can be useful for the methods disclosed herein.

An “Fc receptor” (FcR) refers to a receptor that binds to the Fc region of an antibody. In some embodiments, an FcR is a native human FcR. In some embodiments, an FcR is one which binds an IgG antibody (a gamma receptor) and includes receptors of the FcgRI, FcgRII, and FcgRIII subclasses, including allelic variants and alternatively spliced forms of those receptors. FcgRII receptors include FcgRIIA (an “activating receptor”) and FcgRIIB (an “inhibiting receptor”), which have similar amino acid sequences that differ primarily in the cytoplasmic domains thereof. Activating receptor FcgRIIA contains an immunoreceptor tyrosine-based activation motif (ITAM) in its cytoplasmic domain. Inhibiting receptor FcgRIIB contains an immunoreceptor tyrosine-based inhibition motif (ITIM) in its cytoplasmic domain, (see, e.g., Daeron, Annu. Rev. Immunol. 1997; 15:203-234). FcRs are reviewed, for example, in Ravetch and Kinet, Annu. Rev. Immunol 1991; 9:457-92; Capel et al., Immunomethods 1994; 4:25-34; and de Haas et al., J. Lab. Clin. Med. 1995; 126:330-41. Other FcRs, including those to be identified in the future, are encompassed by the term “Fc receptor” herein. The term “Fc receptor” also includes the neonatal receptor, FcRn, which is responsible for the transfer of maternal IgGs to the fetus (Guyer et al., J. Immunol. 1976; 117:587 and Kim et al., J. Immunol. 1994; 24:249) and regulation of homeostasis of immunoglobulins. Methods of measuring binding to FcRn are known (see, e.g., Ghetie and Ward., Immunol. Today 1997; 18 (12): 592-598; Ghetie et al., Nature Biotechnology, 1997; 15 (7): 637-640; Hinton et al., J. Biol. Chem. 2004; 279 (8): 6213-6216; WO 2004/92219).

An “effector cell” refers to a leukocyte which express one or more FcRs and performs effector functions. In certain embodiments, effector cells express at least FcgRIII and perform ADCC effector function(s). Examples of leukocytes which mediate ADCC include peripheral blood mononuclear cells (PBMC), natural killer (NK) cells, monocytes, macrophages, cytotoxic T cells, and neutrophils. Effector cells may be isolated from a native source, e.g., from blood.

The term “antibody-dependent cell-mediated cytotoxicity” or “ADCC” refers to a form of cytotoxicity in which secreted Ig bound onto Fc receptors (FcRs) present on certain cytotoxic cells (e.g., NK cells, neutrophils, and macrophages) enable these cytotoxic effector cells to bind specifically to an antigen-bearing target cell and subsequently kill the target cell with cytotoxins. The primary cells for mediating ADCC, NK cells, express FcgRIII only, whereas monocytes express FcgRI, FcgRII, and FcgRIII. To assess ADCC activity of a molecule of interest, an in vitro ADCC assay, such as that described in U.S. Pat. Nos. 5,500,362, 5,821,337 or 6,737,056, may be performed. Useful effector cells for such assays include PBMC and NK cells. Alternatively, or additionally, ADCC activity of the molecule of interest may be assessed in vivo, e.g., in an animal model such as that disclosed in Clynes et al., Proc. Natl. Acad. Sci. (USA) 1998; 95:652-656. Additional antibodies with altered Fc region amino acid sequences and increased or decreased ADCC activity are described, e.g., in U.S. Pat. Nos. 7,923,538, and 7,994,290.

The term “enhanced ADCC activity” refers to an antibody that is more effective at mediating ADCC in vitro or in vivo compared to the parent antibody, wherein the antibody and the parent antibody differ in at least one structural aspect, and when the amounts of such antibody and parent antibody used in the assay are essentially the same. In some embodiments, the antibody and the parent antibody have the same amino acid sequence, but the antibody is afucosylated while the parent antibody is fucosylated. In some embodiments, ADCC activity will be determined using an in vitro ADCC assay, but other assays or methods for determining ADCC activity, e.g. in an animal model etc., are contemplated. In some embodiments, an antibody with enhanced ADCC activity has enhanced affinity for FcgRIIIA.

The term “altered” FcR binding affinity or ADCC activity refers to an antibody which has either enhanced or diminished activity for one or more of FcR binding activity or ADCC activity compared to a parent antibody, wherein the antibody and the parent antibody differ in at least one structural aspect. An antibody that “displays increased binding” to an FcR binds at least one FcR with better affinity than the parent antibody. An antibody that “displays decreased binding” to an FcR, binds at least one FcR with lower affinity than a parent antibody. Such antibodies that display decreased binding to an FcR may possess little or no appreciable binding to an FcR, e.g., 0-20 percent binding to the FcR compared to a native sequence IgG Fc region.

The term “Complement dependent cytotoxicity” or “CDC” refers to the lysis of a target cell in the presence of complement. Activation of the classical complement pathway is initiated by the binding of the first component of the complement system (C1q) to antibodies (of the appropriate subclass), which are bound to their cognate antigen. To assess complement activation, a CDC assay, e.g., as described in Gazzano-Santoro et al., J. Immunol. Methods 1996; 202:163, may be performed. Antibodies with altered Fc region amino acid sequences and increased or decreased C1q binding capability are described, e.g., in U.S. Pat. Nos. 6,194,551, 7,923,538, 7,994,290 and WO 1999/51642.

A “host cell” refers to an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) of this invention.

A “vector” refers to a construct, which is capable of delivering, and, preferably, expressing, one or more gene(s) or sequence(s) of interest (e.g. an antibody-encoding gene) in a host cell. Examples of vectors include, but are not limited to plasmids and viral vectors, and may include naked nucleic acids, or may include nucleic acids associated with delivery-aiding materials (e.g. cationic condensing agents, liposomes, etc). Vectors may include DNA or RNA. An “expression vector” as used herein refers to a vector that includes at least one polypeptide-encoding gene, at least one regulatory element (e.g. promoter sequence, poly (A) sequence) relating to the transcription or translation of the gene. Typically, a vector used herein contains at least one antibody-encoding gene, as well as one or more of regulatory elements or selectable markers. Vector components may include, for example, one or more of the following: a signal sequence; an origin of replication; one or more marker genes; suitable transcriptional controlling elements (such as promoters, enhancers and terminator). For translation, one or more translational controlling elements may also be included such as ribosome binding sites, translation initiation sites, and stop codons.

An “isolated” molecule (e.g. antibody) refers to a molecule that by virtue of its origin or source of derivation (1) is not associated with naturally associated components that accompany it in its native state, (2) is substantially free of other molecules from the same source, e.g., species, cell from which it is expressed, library, etc., (3) is expressed by a cell from a different species, or (4) does not occur in nature. Thus, a molecule that is chemically synthesized, or expressed in a cellular system different from the system from which it naturally originates, will be “isolated” from its naturally associated components. A molecule also may be rendered substantially free of naturally associated components by isolation, using purification techniques well known in the art.

A “polypeptide” or “protein” (used interchangeably herein) refers to a chain of amino acids of any length. The chain may be linear or branched. The chain may comprise one or more of modified amino acids. The terms also encompass an amino acid chain that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that the polypeptides can occur as single chains or associated chains.

A “polynucleotide” or “nucleic acid,” (used interchangeably herein) refers to a chain of nucleotides of any length, and includes DNA and RNA. The nucleotides can be deoxyribonucleotides, ribonucleotides, modified nucleotides or bases or their analogs, or any substrate that can be incorporated into a chain by DNA or RNA polymerase. A polynucleotide may comprise modified nucleotides, such as methylated nucleotides and their analogs. If present, modification to the nucleotide structure may be imparted before or after assembly of the chain. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, alpha- or beta-anomeric sugars, epimeric sugars such as arabinose, xyloses or lyxoses, pyranose sugars, furanose sugars, sedoheptuloses, acyclic analogs and abasic nucleoside analogs such as methyl riboside.

A “conservative substitution” refers to replacement of one amino acid by a biologically, chemically or structurally similar residue. Biologically similar means that the substitution does not destroy a biological activity. Structurally similar means that the amino acids have side chains with similar length, such as alanine, glycine and serine or a similar size. Chemical similarity means that the residues have the same charge or are both hydrophilic or hydrophobic. Particular examples include the substitution of a hydrophobic residue, such as isoleucine, valine, leucine or methionine with another, or the substitution of one polar residue for another, such as the substitution of arginine for lysine, glutamic acid for aspartic acid or glutamine for asparagine, serine for threonine, and the like. Particular examples of conservative substitutions include the substitution of a hydrophobic residue such as isoleucine, valine, leucine or methionine for one another, the substitution of a polar residue for another, such as the substitution of arginine for lysine, glutamic acid for aspartic acid, or glutamine for asparagine, and the like. Conservative amino acid substitutions typically include, for example, substitutions within the following groups: glycine, alanine, valine, isoleucine, leucine; aspartic acid, glutamic acid; asparagine, glutamine; serine, threonine; lysine, arginine; and phenylalanine, tyrosine.

The term “identity” or “identical to” refers to the overall relatedness between polymeric molecules, e.g., between nucleic acid molecules (e.g., DNA molecules or RNA molecules) or between polypeptide molecules. “Identity” measures the percent of identical matches between two or more sequences with gap alignments addressed by a particular mathematical model of computer programs (e.g. algorithms), which are well known in the art.

The terms “increase,” improve,” “decrease” or “reduce” refer to values that are relative to a baseline measurement, such as a measurement in the same individual prior to initiation of treatment described herein, or a measurement in a control individual or subject (or multiple control individuals or subjects) in the absence of the treatment described herein. In some embodiments, a “control individual” is an individual afflicted with the same form of disease or injury as an individual being treated. In some embodiments, a “control individual” is an individual that is not afflicted with the same form of disease or injury as an individual being treated.

The term ‘excipient’ refers to any material which, which combined with an active ingredient of interest (e.g. antibody), allow the active ingredient to retain biological activity. The choice of excipient will to a large extent depend on factors such as the mode of administration, the effect of the excipient on solubility and stability, and the nature of the dosage form. As used herein, “excipient” “includes any and all solvents, dispersion media, coatings, antibacterial and antifungal agents, isotonic and absorption delaying agents, carriers, diluents and the like that are physiologically compatible. Examples of an excipient include one or more of water, saline, phosphate buffered saline, dextrose, glycerol, ethanol and the like, as well as combinations thereof, and may include isotonic agents, for example, sugars, sodium chloride, or polyalcohols such as mannitol, or sorbitol in the composition.

The terms “treating”, “treat” or “treatment” refer to any type of treatment, e.g. such as to relieve, alleviate, or slow the progression of the patient's disease, disorder or condition or any tissue damage associated with the disease. In some embodiments, the disease, disorder or condition is cancer.

The terms “prevent” or “prevention” refer to one or more of delay of onset, reduction in frequency, or reduction in severity of at least one sign or symptom (e.g., size of tumor) of a particular disease, disorder or condition (e.g., cancer). In some embodiments, prevention is assessed on a population basis such that an agent is considered to “prevent” a particular disease, disorder or condition if a statistically significant decrease in the development, frequency or intensity of one or more symptoms of the disease, disorder or condition is observed in a population susceptible to the disease, disorder or condition. Prevention may be considered complete when onset of disease, disorder or condition has been delayed for a predefined period of time.

The terms “subject, “individual” or “patient,” (used interchangeably herein), refer to any animal, including mammals. Mammals according to the invention include canine, feline, bovine, caprine, equine, ovine, porcine, rodents, lagomorphs, primates, humans and the like, and encompass mammals in utero. In an embodiment, humans are suitable subjects. Human subjects may be of any gender and at any stage of development. In some embodiments, a subject is a patient with disease ***.

The term “therapeutically effective amount” refers to the amount of active ingredient that elicits the biological or medicinal response in a tissue, system, animal, individual or human that is being sought by a researcher, veterinarian, medical doctor or other clinician, which may include one or more of the following:
(1) preventing the disease; for example, preventing a disease, condition or disorder in an individual that may be predisposed to the disease, condition or disorder but does not yet experience or display the pathology or symptomatology of the disease;
(2) inhibiting the disease; for example, inhibiting a disease, condition or disorder in an individual that is experiencing or displaying the pathology or symptomatology of the disease, condition or disorder (i.e., arresting or slowing further development of the pathology or symptomatology); and
(3) ameliorating the disease; for example, ameliorating a disease, condition or disorder in an individual that is experiencing or displaying the pathology or symptomatology of the disease, condition or disorder (i.e., reversing the pathology or symptomatology).

### Anti-LILRB1 Antibodies, Anti-LILRB2 Antibodies and LILRB1×LILRB2 Bispecific Antibodies

Leukocyte immunoglobulin-like receptor B1 (LILRB1) and B2 (LILRB2) are negative signaling immune receptors that are expressed on the surface of myeloid-derived suppressive cells (MDSCs), dendritic cells (DCs), monocytes and macrophages. It is believed that blocking LILRB1, LILRB2, or both LILRB2 and LILRB2 interaction with Human Leukocyte antigen G (HLA-G) with an antagonist antibody or an antagonist bispecific antibody may limit myeloid/lymphoid-induced immune suppression and thereby promote anti-tumor immunity.

As used herein, the term LILRB1 and LILRB2, includes variants, isoforms, homologs, orthologs and paralogs of LILRB1 and LILRB2, respectively. In some embodiments, an antibody disclosed herein cross-reacts with LILRB1 or LILRB2, respectively, from species other than human, such as LILRB1 or LILRB2 of cynomolgus monkey, as well as different forms of LILRB1 or LILRB2, respectively. In some embodiments, an antibody may be completely specific for human LILRB1 or LILRB2 and may not exhibit species cross-reactivity (e.g., does not bind mouse LILRB1 or LILRB2) or other types of cross-reactivity. As used herein the term LILRB1 and LILRB2 refers to naturally occurring human LILRB1 and LILRB2, respectively, unless contextually dictated otherwise. Therefore, an “LILRB1 antibody”, “LILRB2 antibody” “anti-LILRB1 antibody”, “anti-LILRB2 antibody”, or other similar designation means any antibody (as defined herein) that binds or reacts with LILRB1 or LILRB2, respectively, an isoform, fragment or derivative thereof, respectively.

Without wishing to be bound by any particular theory, blockade of LILRB1 and HLA-G interaction inhibits LILRB1 signaling, blockade of LILRB2 and HLA-G interaction inhibits LILRB2 signaling.

A neutralizing or “blocking” antibody or bispecific antibody refers to an antibody or bispecific antibody whose binding to LILRB1, LILRB2, or both respectively, (i) interferes with, limits, or inhibits the interaction between LILRB1 and a LILRB1 ligand, between LILRB2 and a LILRB2 ligand, such as HLA-G, or both; or (ii) results in inhibition of at least one biological function of LILRB1 and LILRB2 signaling, or both. Assays to determine neutralization by an antibody of the disclosure are well-known in the art.

“Biological function” or “biological activity” of LILRB1 and LILRB2, respectively, is meant to include the binding between LILRB1 and its ligand HLA-G and LILRB2 and its ligand HLA-G, respectively, any HLA-G dependent LILRB1 signaling and LILRB2 signaling, respectively. The biological function or biological activity of LILRB1 and LILRB2, respectively, can, but need not be, mediated by the interaction between LILRB1 and its ligands, and LILRB2 and its ligand, respectively, and in particular HLA-G.

A bispecific antibody that comprises one arm that binds to LILRB1 (“B1 arm”) and another arm that binds to LILRB2 (“B2 arm”), is described herein as a LILRB1×LILRB2 bispecific antibody. In Table 1, Table 2, and in Table 3, sequences for LILRB1×LILRB2 bispecific antibodies BsAb-1882 and BsAb-1880, anti-LRLIB1 antibody Ab-B1-1704 and anti-LILRB2 antibody Ab-B2-1825 are provided, respectively. The CDRs within the VH and VL are marked: For VH CDR1 and VH CDR2, the underlined sequences are Kabat CDR sequences, and the bolded and italic sequences are Chothia CDR sequences. For VH CDR3 and VL CDR1, VL CDR2 and VL CDR3, the Kabat CDR sequences and the respective Chothia CDR sequences are identical and are marked by underlines.

TABLE 1
Amino Acid Sequence of LILRB1 x LILRB2 Bispecific Antibodies BsAb-
1882 and BsAb-1880

| | |
|---|---|
| BsAb-1882 | EVQLVESGGG LVQPGGSLRL SCAASGFTFS SYAMHWVRQA |
| BsAb-1880 | PGKGLEWVAV ISYDGSNKYY ADSVKGRFTI SRDNAKNSLY |
| B1 arm | LQMNSLRAED TAVYYCARDK GIGTSPLDYW |
| VH | GQGTLVTVSS |
| | (SEQ ID NO: 16) |
| BsAb-1882 | SYAMH (SEQ ID NO: 9, Kabat) |
| BsAb-1880 | GFTFSSY (SEQ ID NO: 10, Chothia) |
| B1 arm | GFTFSSYAMH (SEQ ID NO: 11, extended) |
| VH CDR1 | |
| BsAb-1882 | V ISYDGSNKYY ADSVKG (SEQ ID NO: 12, Kabat) |
| BsAb-1880 | SYDGSN (SEQ ID NO: 13, Chothia) |
| B1 arm | |
| VH CDR2 | |
| BsAb-1882 | DK GIGTSPLDY (SEQ ID NO: 14) |
| BsAb-1880 | |
| B1 arm | |
| VH CDR3 | |
| BsAb-1882 | SYELTQPPSV SVSPGQTASI TCGGNNIGSK GVHWYQQKPG |
| BsAb-1880 | QSPVLVISYD SDRPSGIPER FSGSNSGNTA TLTISGTQAM |
| B1 arm VL | DEADYYCQVW DSSTRVFGTG TKVTVL |
| | (SEQ ID NO: 6) |
| BsAb-1882 | GGNNIGSK GVH (SEQ ID NO: 2) |
| BsAb-1880 | |
| B1 arm | |
| VL CDR1 | |
| BsAb-1882 | YD SDRPS (SEQ ID NO: 3) |
| BsAb-1880 | |
| B1 arm | |
| VL CDR2 | |
| BsAb-1882 | QVW DSSTRV (SEQ ID NO: 4) |
| BsAb-1880 | |
| B1 arm | |
| VL CDR3 | |
| BsAb-1882 | EVQLVESGGG LVQPGGSLRL SCAASGFTFS SYAMHWVRQA |
| B1 arm | PGKGLEWVAV ISYDGSNKYY ADSVKGRFTI SRDNAKNSLY |
| Heavy chain | LQMNSLRAED TAVYYCARDK GIGTSPLDYW GQGTLVTVSS |
| (VH-CH1) | ASTKGPSVFP LAPSSKSTSG GTAALGCLVK DYFPEPVTVS |
| | WNSGALTSGV HTFPAVLQSS GLYSLSSVVT VPSSSLGTQT |
| | YICNVNHKPS NTKVDKKVEP KSC |
| | (SEQ ID NO: 8) |
| BsAb-1882 | SYELTQPPSV SVSPGQTASI TCGGNNIGSK GVHWYQQKPG |
| B1 arm | QSPVLVISYD SDRPSGIPER FSGSNSGNTA TLTISGTQAM |
| Light chain | DEADYYCQVW DSSTRVFGTG TKVTVLGQPK AAPSVTLFPP |
| (VL-CL-hinge- | SSEELQANKA TLVCLISDFY PGAVTVAWKA DSSPVKAGVE |
| Fc) | TTTPSKQSNN KYAASSYLSL TPEQWKSHRS YSCQVTHEGS |
| | TVEKTVAPTE CSDKTHTCPP CPAPEAAGAP SVFLFPPKPK |
| | DTLMISRTPE VTCVVVDVSH EDPEVKFNWY VDGVEVHNAK |
| | TKPREEQYNS TYRVVSVLTV LHQDWLNGKE YKCKVSNKAL |
| | PAPIEKTISK AKGQPREPQV YTLPPCREEM TKNQVSLSCA |
| | VKGFYPSDIA VEWESNGQPE NNYKTTPPVL DSDGSFFLVS |
| | KLTVDKSRWQ QGNVFSCSVM HEALHNHYTQ KSLSLSPGK |
| | (SEQ ID NO: 1) |
| BsAb-1882 | SYELTQPPSV SVSPGQTASI TCGGNNIGSK GVHWYQQKPG |
| B1 arm | QSPVLVISYD SDRPSGIPER FSGSNSGNTA TLTISGTQAM |
| Light chain | DEADYYCQVW DSSTRVFGTG TKVTVLGQPK AAPSVTLFPP |
| Without C | SSEELQANKA TLVCLISDFY PGAVTVAWKA DSSPVKAGVE |
| terminal lysine | TTTPSKQSNN KYAASSYLSL TPEQWKSHRS YSCQVTHEGS |
| | TVEKTVAPTE CSDKTHTCPP CPAPEAAGAP SVFLFPPKPK |
| | DTLMISRTPE VTCVVVDVSH EDPEVKFNWY VDGVEVHNAK |
| | TKPREEQYNS TYRVVSVLTV LHQDWLNGKE YKCKVSNKAL |
| | PAPIEKTISK AKGQPREPQV YTLPPCREEM TKNQVSLSCA |
| | VKGFYPSDIA VEWESNGQPE NNYKTTPPVL DSDGSFFLVS |
| | KLTVDKSRWQ QGNVFSCSVM HEALHNHYTQ KSLSLSPG |
| | (SEQ ID NO: 35) |
| BsAb-1882 | EVQLLESGGG LVQPGGSLRL SCAASGFTFS SYAMSWVRQA |
| BsAb-1880 | PGKGLEWVSA ISGSGGSTYY ADSVKGRFTI SRDNSKNTLY |
| B2 arm VH | LQMNSLRAED TAVYYCWRWL NLGDAFDIWG QGTMVTVSS |
| | (SEQ ID NO: 31) |
| BsAb-1882 | SYAMS (SEQ ID NO: 24, Kabat) |
| BsAb-1880 | GFTFSSY (SEQ ID NO: 25, Chothia) |
| B2 arm | GFTFSSYAMS (SEQ ID NO: 26, extended) |
| VH CDR1 | |
| BsAb-1882 | A ISGSGGSTYY ADSVKG (SEQ ID NO: 27, Kabat) |
| BsAb-1880 | SGSGGS (SEQ ID NO: 28, Chothia) |
| B2 arm | |
| VH CDR2 | |
| BsAb-1882 | WLNLGDAFDI (SEQ ID NO: 29) |
| BsAb-1880 | |
| B2 arm | |
| VH CDR3 | |
| BsAb-1882 | QSVLTQPPSA SGTPGQRVTI SCTGSSSNIG SNTVNWYQQL |
| BsAb-1880 | PGTAPKLLIY SDNQRPSGVP DRFSGSTSGT SASLAISGLR |
| B2 arm VL | SEDEADYFCA TWEDVLAGYV VFGGGTKLTV L |
| | (SEQ ID NO: 22) |
| BsAb-1882 | TGSSSNIG SNTVN (SEQ ID NO: 18) |
| BsAb-1880 | |
| B2 arm | |
| VL CDR1 | |
| BsAb-1882 | SDNQRPS (SEQ ID NO: 19) |
| BsAb-1880 | |
| B2 arm | |
| VL CDR2 | |
| BsAb-1882 | ATWEDVLAGYVV (SEQ ID NO: 20) |
| BsAb-1880 | |
| B2 arm | |
| VL CDR3 | |
| BsAb-1882 | EVQLLESGGG LVQPGGSLRL SCAASGFTFS SYAMSWVRQA |
| B2 arm | PGKGLEWVSA ISGSGGSTYY ADSVKGRFTI SRDNSKNTLY |
| Heavy chain | LQMNSLRAED TAVYYCWRWL NLGDAFDIWG QGTMVTVSSA |
| | STKGPSVFPL APSSKSTSGG TAALGCLVKD YFPEPVTVSW |
| | NSGALTSGVH TFPAVLQSSG LYSLSSVVTV PSSSLGTQTY |
| | ICNVNHKPSN TKVDKKVEPK SCDKTHTCPP CPAPEAAGAP |
| | SVFLFPPKPK DTLMISRTPE VTCVVVDVSH EDPEVKFNWY |
| | VDGVEVHNAK TKPREEQYNS TYRVVSVLTV LHQDWLNGKE |
| | YKCKVSNKAL PAPIEKTISK AKGQPREPQV CTLPPSREEM |
| | TKNQVSLWCL VKGFYPSDIA VEWESNGQPE NNYKTTPPVL |
| | DSDGSFFLYS KLTVDKSRWQ QGNVFSCSVM HEALHNHYTQ |
| | KSLSLSPGK |
| | (SEQ ID NO: 23) |
| BsAb-1882 | EVQLLESGGG LVQPGGSLRL SCAASGFTFS SYAMSWVRQA |
| B2 arm | PGKGLEWVSA ISGSGGSTYY ADSVKGRFTI SRDNSKNTLY |
| Heavy chain | LQMNSLRAED TAVYYCWRWL NLGDAFDIWG QGTMVTVSSA |
| without C | STKGPSVFPL APSSKSTSGG TAALGCLVKD YFPEPVTVSW |
| terminal lysine | NSGALTSGVH TFPAVLQSSG LYSLSSVVTV PSSSLGTQTY |
| | ICNVNHKPSN TKVDKKVEPK SCDKTHTCPP CPAPEAAGAP |
| | SVFLFPPKPK DTLMISRTPE VTCVVVDVSH EDPEVKFNWY |
| | VDGVEVHNAK TKPREEQYNS TYRVVSVLTV LHQDWLNGKE |
| | YKCKVSNKAL PAPIEKTISK AKGQPREPQV CTLPPSREEM |
| | TKNQVSLWCL VKGFYPSDIA VEWESNGQPE NNYKTTPPVL |
| | DSDGSFFLYS KLTVDKSRWQ QGNVFSCSVM HEALHNHYTQ |
| | KSLSLSPG |
| | (SEQ ID NO: 36) |
| BsAb-1882 | QSVLTQPPSA SGTPGQRVTI SCTGSSSNIG SNTVNWYQQL |
| BsAb-1880 | PGTAPKLLIY SDNQRPSGVP DRFSGSTSGT SASLAISGLR |
| B2 arm | SEDEADYFCA TWEDVLAGYV VFGGGTKLTV LGQPKAAPSV |
| Light chain | TLFPPSSEEL QANKATLVCL ISDFYPGAVT VAWKADSSPV |
| | KAGVETTTPS KQSNNKYAAS SYLSLTPEQW KSHRSYSCQV |
| | THEGSTVEKT VAPTECS |
| | (SEQ ID NO: 17) |
| BsAb-1880 | SYELTQPPSV SVSPGQTASI TCGGNNIGSK GVHWYQQKPG |
| B1 arm | QSPVLVISYD SDRPSGIPER FSGSNSGNTA TLTISGTQAM |
| Light chain | DEADYYCQVW DSSTRVFGTG TKVTVLGQPK AAPSVTLFPP |
| | SSEELQANKA TLVCLISDFY PGAVTVAWKA DSSPVKAGVE |
| | TTTPSKQSNN KYAASSYLSL TPEQWKSHRS YSCQVTHEGS |
| | TVEKTVAPTE CS |
| | (SEQ ID NO: 40) |
| BsAb-1880 | EVQLVESGGG LVQPGGSLRL SCAASGFTFS SYAMHWVRQA |
| B1 arm | PGKGLEWVAV ISYDGSNKYY ADSVKGRFTI SRDNAKNSLY |
| Heavy chain | LQMNSLRAED TAVYYCARDK GIGTSPLDYW GQGTLVTVSS |
| | ASTKGPSVFP LAPSSKSTSG GTAALGCLVK DYFPEPVTVS |
| | WNSGALTSGV HTFPAVLQSS GLYSLSSVVT VPSSSLGTQT |
| | YICNVNHKPS NTKVDKKVEP KSCRKTHTCP PCPAPEAAGA |
| | PSVFLFPPKP KDTLMISRTP EVTCVVVDVS HEDPEVKFNW |
| | YVDGVEVHNA KTKPREEQYN STYRVVSVLT VLHQDWLNGK |
| | EYKCKVSNKA LPAPIEKTIS KAKGQPREPQ VYTLPPSREE |
| | MTKNQVSLTC LVKGFYPSDI AVEWESNGQP ENNYKTTPPV |
| | LDSDGSFFLY SRLTVDKSRW QQGNVFSCSV MHEALHNHYT |
| | QKSLSLSPGK |
| | (SEQ ID NO: 41) |
| BsAb-1880 | EVQLLESGGG LVQPGGSLRL SCAASGFTFS SYAMSWVRQA |
| B2 arm | PGKGLEWVSA ISGSGGSTYY ADSVKGRFTI SRDNSKNTLY |
| Heavy chain | LQMNSLRAED TAVYYCWRWL NLGDAFDIWG QGTMVTVSSA |
| | STKGPSVFPL APSSKSTSGG TAALGCLVKD YFPEPVTVSW |
| | NSGALTSGVH TFPAVLQSSG LYSLSSVVTV PSSSLGTQTY |
| | ICNVNHKPSN TKVDKKVEPK SCEKTHTCPP CPAPEAAGAP |
| | SVFLFPPKPK DTLMISRTPE VTCVVVDVSH EDPEVKFNWY |
| | VDGVEVHNAK TKPREEQYNS TYRVVSVLTV LHQDWLNGKE |
| | YKCKVSNKAL PAPIEKTISK AKGQPREPQV YTLPPSREEM |
| | TKNQVSLTCE VKGFYPSDIA VEWESNGQPE NNYKTTPPVL |
| | DSDGSFFLYS KLTVDKSRWQ QGNVFSCSVM HEALHNHYTQ |
| | KSLSLSPGK |
| | (SEQ ID NO: 42) |

TABLE 2
Amino Acid Sequences of anti huLILRB1 antibody Ab-B1-1704

| | |
|---|---|
| Ab-B1-1704 | EVQLVESGGG LVQPGGSLRL SCAASGFTFS SYAMHWVRQA |
| VH | PGKGLEWVAV ISYDGSNKYY ADSVKGRFTI SRDNAKNSLY |
| | LQMNSLRAED TAVYYCARDK GIGTSPLDYW |
| | GQGTLVTVSS |
| | (SEQ ID NO: 16) |
| Ab-B1-1704 | SYAMH (SEQ ID NO: 9, Kabat) |
| VH CDR1 | GFTFSSY (SEQ ID NO: 10, Chothia) |
| | GFTFSSYAMH (SEQ ID NO: 11, extended) |
| Ab-B1-1704 | V ISYDGSNKYY ADSVKG (SEQ ID NO: 12, Kabat) |
| VH CDR2 | SYDGSN (SEQ ID NO: 13, Chothia) |
| Ab-B1-1704 | DK GIGTSPLDY (SEQ ID NO: 14) |
| VH CDR3 | |
| Ab-B1-1704 | SYELTQPPSV SVSPGQTASI TCGGNNIGSK GVHWYQQKPG |
| VL | QSPVLVISYD SDRPSGIPER FSGSNSGNTA TLTISGTQAM |
| | DEADYYCQVW DSSTRVFGTG TKVTVL |
| | (SEQ ID NO: 6) |
| Ab-B1-1704 VL | GGNNIGSK GVH (SEQ ID NO: 2) |
| CDR1 | |
| Ab-B1-1704 VL | YD SDRPS (SEQ ID NO: 3) |
| CDR2 | |
| Ab-B1-1704 VL | QVW DSSTRV (SEQ ID NO: 4) |
| CDR3 | |
| Ab-B1-1704 | EVQLVESGGG LVQPGGSLRL SCAASGFTFS SYAMHWVRQA |
| Heavy chain | PGKGLEWVAV ISYDGSNKYY ADSVKGRFTI SRDNAKNSLY |
| | LQMNSLRAED TAVYYCARDK GIGTSPLDYW GQGTLVTVSS |
| | ASTKGPSVFP LAPSSKSTSG GTAALGCLVK DYFPEPVTVS |
| | WNSGALTSGV HTFPAVLQSS GLYSLSSVVT VPSSSLGTQT |
| | YICNVNHKPS NTKVDKKVEP KSCDKTHTCP PCPAPEAAGA |
| | PSVFLFPPKP KDTLMISRTP EVTCVVVDVS HEDPEVKFNW |
| | YVDGVEVHNA KTKPREEQYN STYRVVSVLT VLHQDWLNGK |
| | EYKCKVSNKA LPAPIEKTIS KAKGQPREPQ VYTLPPSREE |
| | MTKNQVSLTC LVKGFYPSDI AVEWESNGQP ENNYKTTPPV |
| | LDSDGSFFLY SKLTVDKSRW QQGNVFSCSV MHEALHNHYT |
| | QKSLSLSPGK |
| | (SEQ ID NO: 38) |
| Ab-B1-1704 | SYELTQPPSV SVSPGQTASI TCGGNNIGSK GVHWYQQKPG |
| Light chain | QSPVLVISYD SDRPSGIPER FSGSNSGNTA TLTISGTQAM |
| | DEADYYCQVW DSSTRVFGTG TKVTVLGQPK AAPSVTLFPP |
| | SSEELQANKA TLVCLISDFY PGAVTVAWKA DSSPVKAGVE |
| | TTTPSKQSNN KYAASSYLSL TPEQWKSHRS YSCQVTHEGS |
| | TVEKTVAPTE CS |
| | (SEQ ID NO: 37) |

TABLE 3
Amino Acid Sequences of anti huLILRB2 antibody Ab-B2-1825

| | |
|---|---|
| Ab-B2-1825 | EVQLLESGGG LVQPGGSLRL SCAASGFTFS SYAMSWVRQA |
| Ab-B2-1825 | PGKGLEWVSA ISGSGGSTYY ADSVKGRFTI SRDNSKNTLY |
| VH | LQMNSLRAED TAVYYCWRWL NLGDAFDIWG QGTMVTVSS |
| | (SEQ ID NO: 31) |
| Ab-B2-1825 | SYAMS (SEQ ID NO: 24, Kabat) |
| VH CDR1 | GFTFSSY (SEQ ID NO: 25, Chothia) |
| | GFTFSSYAMS (SEQ ID NO: 26, extended) |
| Ab-B2-1825 | A ISGSGGSTYY ADSVKG (SEQ ID NO: 27, Kabat) |
| VH CDR2 | SGSGGS (SEQ ID NO: 28, Chothia) |
| Ab-B2-1825 | WLNLGDAFDI (SEQ ID NO: 29) |
| VH CDR3 | |
| Ab-B2-1825 | QSVLTQPPSA SGTPGQRVTI SCTGSSSNIG SNTVNWYQQL |
| VL | PGTAPKLLIY SDNQRPSGVP DRFSGSTSGT SASLAISGLR |
| | SEDEADYFCA TWEDVLAGYV VFGGGTKLTV L |
| | (SEQ ID NO: 22) |
| Ab-B2-1825 | TGSSSNIG SNTVN (SEQ ID NO: 18) |
| VL CDR1 | |
| Ab-B2-1825 | SDNQRPS (SEQ ID NO: 19) |
| VL CDR2 | |
| Ab-B2-1825 | ATWEDVLAGYVV (SEQ ID NO: 20) |
| VL CDR3 | |
| Ab-B2-1825 | EVQLLESGGG LVQPGGSLRL SCAASGFTFS SYAMSWVRQA |
| Heavy chain | PGKGLEWVSA ISGSGGSTYY ADSVKGRFTI SRDNSKNTLY |
| | LQMNSLRAED TAVYYCWRWL NLGDAFDIWG QGTMVTVSSA |
| | STKGPSVFPL APSSKSTSGG TAALGCLVKD YFPEPVTVSW |
| | NSGALTSGVH TFPAVLQSSG LYSLSSVVTV PSSSLGTQTY |
| | ICNVNHKPSN TKVDKKVEPK SCDKTHTCPP CPAPEAAGAP |
| | SVFLFPPKPK DTLMISRTPE VTCVVVDVSH EDPEVKFNWY |
| | VDGVEVHNAK TKPREEQYNS TYRVVSVLTV LHQDWLNGKE |
| | YKCKVSNKAL PAPIEKTISK AKGQPREPQV YTLPPSREEM |
| | TKNQVSLTCL VKGFYPSDIA VEWESNGQPE NNYKTTPPVL |
| | DSDGSFFLYS KLTVDKSRWQ QGNVFSCSVM HEALHNHYTQ |
| | KSLSLSPGK |
| | (SEQ ID NO: 39) |
| Ab-B2-1825 | QSVLTQPPSA SGTPGQRVTI SCTGSSSNIG SNTVNWYQQL |
| Light chain | PGTAPKLLIY SDNQRPSGVP DRFSGSTSGT SASLAISGLR |
| | SEDEADYFCA TWEDVLAGYV VFGGGTKLTV LGQPKAAPSV |
| | TLFPPSSEEL QANKATLVCL ISDFYPGAVT VAWKADSSPV |
| | KAGVETTTPS KQSNNKYAAS SYLSLTPEQW KSHRSYSCQV |
| | THEGSTVEKT VAPTECS |
| | (SEQ ID No: 17) |

LILRB1×LILRB2 bispecific antibody BsAb-1882 is a human IgG1 antibody that has reduced effector function, and comprising two binding arms, B1 arm and B2 arm, with a knob and hole type of mutations in the Fc region to facilitate the heterodimerization. BsAb-1882 B2 arm has a conventional configuration, i.e., its heavy chain comprises from N terminal to C terminal VH, CH1, an IgG1 hinge region and an IgG1 Fc chain, and its light chain comprises a VL and CL. BsAb-1882 B1 arm however has its Fc chain connected to the C terminus of the light chain CL via a IgG1 hinge region fragment. The BsAb-1882 B1 arm heavy chain comprises from N to C terminus VH and CH1, and its light chain comprises from N to C terminus VL, CL, an IgG1 hinge region fragment and an IgG1 Fc chain (the Fc chain comprising a fragment of the IgG1 hinge and an IgG1 CH2 and an IgG1 CH3).

Knob-into-hole mutations in BsAb-1882 facilitate Fc-region heterodimer formation. Knob mutations on the B2 arm heavy chain are T366W and Y349C (EU numbering, or T389W and Y370C, respectively, using Kabat numbering). Hole mutations on the B1 arm are T366S, L368A and Y407V (EU numbering) or T389S, L391A and Y438V (Kabat numbering) with S354C (EU numbering or S375C by Kabat). The bispecific IgG1 also harbors mutations to reduce effector function (L234A, L235A and G237A; EU numbering or L247A, L248A and G250A; Kabat numbering).

LILRB1×LILRB2 bispecific antibody BsAb-1880 is also a human IgG1 antibody. BsAb-1880 has identical VH and VL of both the B1 and B2 arm as those of BsAb-1882. However, both the B1 arm and B2 arm of BsAb-1880 have conventional configuration, that is the two Fc chains are connected to the C terminus of the CH1 domain of the heavy chains. Also, EE/RR mutations in BsAb-1880 facilitate the bispecific antibody formation.

In BsAb-1880, the B1 arm heavy chain constant region comprises the mutations of D221R and K409R, and the B2 arm heavy chain constant region comprises the mutations of D221E and L368E. BsAb-1880 also harbors mutations to reduce effector function (L234A, L235A and G237A; EU numbering).

The anti-LILRB1 antibody Ab-B1-1704 described in Table 2 and throughout this specification is a human IgG1 antibody unless otherwise specified. The anti-LILRB2 antibody Ab-B2-1825 described in Table 3 and throughout this specification is also a human IgG1 antibody unless otherwise specified.

In some embodiments, a LILRB1×LILRB2 bispecific antibody of the disclosure encompasses an antibody that i) competes for binding to human LILRB1 with HLA-G, and also competes for binding to human LILRB2 with HLA-G, (ii) binds the same epitope on LILRB1 as an anti-LILRB1 antibody comprising a heavy chain variable region having the amino acid sequence set forth in SEQ ID NO: 16, and light chain variable region having the amino acid sequence set forth in SEQ ID NO:6, and iii) binds the same epitope on LILRB2 as an anti-LILRB2 antibody comprising a heavy chain variable region having the amino acid sequence set forth in SEQ ID NO:31, and a light chain variable region having the amino acid sequence set forth in SEQ ID NO:22.

In some embodiments, an anti-LILRB1 antibody of the disclosure encompasses an antibody that i) competes for binding to human LILRB1 with HLA-G, and ii) binds the same epitope on LILRB1 as an anti-LILRB1 antibody comprising a heavy chain variable region having the amino acid sequence set forth in SEQ ID NO:16, and a light chain variable region having the amino acid sequence set forth in SEQ ID NO:6.

In some embodiments, an anti-LILRB2 antibody of the disclosure encompasses an antibody that i) competes for binding to human LILRB2 with HLA-G, and ii) binds the same epitope on LILRB2 as an anti-LILRB2 antibody comprising a heaving chain variable region having the amino acid sequence set forth in SEQ ID NO:31, and a light chain variable region having the amino acid sequence set forth in SEQ ID NO:22.

An anti-LILRB1 antibody, an anti-LILRB2 antibody or a LILRB1×LILRB2 bispecific antibody of the present disclosure can encompass monoclonal antibodies, polyclonal antibodies, antibody fragments (e.g., Fab, Fab′, F(ab′)₂, Fv, Fc, etc.), chimeric antibodies, bispecific antibodies, heteroconjugate antibodies, single chain (ScFv), mutants thereof, fusion proteins comprising an antibody fragment (e.g., a domain antibody), humanized antibodies, and any other modified configuration of the immunoglobulin molecule that comprises an antigen binding site of the required specificity, including glycosylation variants of antibodies, amino acid sequence variants of antibodies, and covalently modified antibodies. The antibodies may be murine, rat, human, or any other origin (including chimeric or humanized antibodies). In some embodiments, An anti-LILRB1 antibody, an anti-LILRB2 antibody or a LILRB1×LILRB2 bispecific antibody is a monoclonal antibody. In some embodiments, an anti-LILRB1 antibody, an anti-LILRB2 antibody or a LILRB1×LILRB2 bispecific antibody is a human or humanized antibody. In some embodiments, an anti-LILRB1 antibody, an anti-LILRB2 antibody or a LILRB1×LILRB2 bispecific antibody is a chimeric antibody.

The invention also provides CDR portions of anti-LILRB1 antibodies, anti-LILRB2 antibodies and LILRB1×LILRB2 bispecific antibodies. Determination of CDR regions is well within the skill of the art. It is understood that in some embodiments, CDRs can be a combination of the Kabat and Chothia CDR (also termed “combined CDRs” or “extended CDRs”). In another approach, referred to herein as the “conformational definition” of CDRs, the positions of the CDRs may be identified as the residues that make enthalpic contributions to antigen binding. See, e.g., Makabe et al., 2008, Journal of Biological Chemistry, 283:1156-1166. In general, “conformational CDRs” include the residue positions in the Kabat CDRs and Vernier zones which are constrained in order to maintain proper loop structure for the antibody to bind a specific antigen. Determination of conformational CDRs is well within the skill of the art. In some embodiments, the CDRs are the Kabat CDRs. In other embodiments, the CDRs are the Chothia CDRs. In other embodiments, the CDRs are the extended, AbM, conformational, or contact CDRs. In other words, in embodiments with more than one CDR, the CDRs may be any of Kabat, Chothia, extended, AbM, conformational, contact CDRs or combinations thereof.

In some embodiments, the LILRB1×LILRB2 bispecific antibody of the disclosure comprises (i) the B1 arm and B2 arm heavy chain CDRs (VH CDR1, CH CDR2 and VH CDR3) of that of BsAb-1882 as shown in Table 1, (ii) the B1 arm and B2 arm light chain CDRs (VL CDR1, VL CDR2 and VL CDR3) of that of BsAb-1882, or (iii) both (i) and (ii).

In some embodiments, anti-LILRB1 antibody of the disclosure comprises (i) the heavy chain CDRs of Ab-B1-1704 as shown in Table 2, (ii) the light chain CDRs of Ab-B1-1704 as shown in Table 2, or (iii) both (i) and (ii).

In some embodiments, anti-LILRB2 antibody of the disclosure comprises (i) the heavy chain CDRs of Ab-B2-1825 as shown in Table 3, (ii) the light chain CDRs of Ab-B2-1825 as shown in Table 3, or (iii) both (i) and (ii).

In some embodiments, an LILRB1×LILRB2 bispecific antibody, an anti-LILRB1 antibody, and an anti-LILRB2 antibody described herein comprises an Fc domain. The Fc domain can be derived from IgA (e.g., IgA₁ or IgA₂), IgG, IgE, or IgG (e.g., IgG₁, IgG₂, IgG₃, or IgG₄). In some embodiments, the Fc domain is a human IgG1 Fc domain.

The invention encompasses modifications to the variable regions, the CDRs and the heavy chain and light chain sequences shown in Table 1 and Table 2. For example, the invention includes antibodies comprising functionally equivalent variable regions and CDRs which do not significantly affect their properties as well as variants which have enhanced or decreased activity or affinity. For example, the amino acid sequence may be mutated to obtain an antibody with the desired binding affinity to LILRB1 or LILRB2. Modification of polypeptides is routine practice in the art and need not be described in detail herein. Examples of modified polypeptides include polypeptides with conservative substitutions of amino acid residues, one or more deletions or additions of amino acids which do not significantly deleteriously change the functional activity, or which mature (enhance) the affinity of the polypeptide for its ligand, or use of chemical analogs

A modification or mutation may also be made in a framework region or constant region to increase the half-life of an antibody provided herein. See, e.g., PCT Publication No. WO 00/09560. A mutation in a framework region or constant region can also be made to alter the immunogenicity of the antibody, to provide a site for covalent or non-covalent binding to another molecule, or to alter such properties as complement fixation, FcR binding and antibody-dependent cell-mediated cytotoxicity. In some embodiments, no more than one to five conservative amino acid substitutions are made within the framework region or constant region. In other embodiments, no more than one to three conservative amino acid substitutions are made within the framework region or constant region. According to the invention, a single antibody may have mutations in any one or more of the CDRs or framework regions of the variable domain or in the constant region.

In some embodiments, the antibody comprises a modified constant region that has increased or decreased binding affinity to a human Fc gamma receptor, is immunologically inert or partially inert, e.g., does not trigger complement mediated lysis, does not stimulate antibody-dependent cell mediated cytotoxicity (ADCC), or does not activate microglia; or has reduced activities (compared to the unmodified antibody) in any one or more of the following: triggering complement mediated lysis, stimulating ADCC, or activating microglia. Different modifications of the constant region may be used to achieve optimal level or combination of effector functions. See, for example, Morgan et al., Immunology 86:319-324, 1995; Lund et al., J. Immunology 157:4963-9 157:4963-4969, 1996; Idusogie et al., J. Immunology 164:4178-4184, 2000; Tao et al., J. Immunology 143:2595-2601, 1989; and Jefferis et al., Immunological Reviews 163:59-76, 1998. In some embodiments, the constant region is modified as described in Eur. J. Immunol., 1999, 29:2613-2624; PCT Publication No. WO99/058572.

Modifications also include glycosylated and nonglycosylated polypeptides, as well as polypeptides with other post-translational modifications, such as, for example, glycosylation with different sugars, acetylation, and phosphorylation. Antibodies are glycosylated at conserved positions in their constant regions (Jefferis and Lund, 1997, Chem. Immunol. 65:111-128; Wright and Morrison, 1997, TibTECH 15:26-32). The oligosaccharide side chains of the immunoglobulins affect the protein's function (Boyd et al., 1996, Mol. Immunol. 32:1311-1318; Wittwe and Howard, 1990, Biochem. 29:4175-4180) and the intramolecular interaction between portions of the glycoprotein, which can affect the conformation and presented three-dimensional surface of the glycoprotein (Jefferis and Lund, supra; Wyss and Wagner, 1996, Current Opin. Biotech. 7:409-416).

Oligosaccharides may also serve to target a given glycoprotein to certain molecules based upon specific recognition structures. Glycosylation of antibodies has also been reported to affect antibody-dependent cellular cytotoxicity (ADCC). In particular, antibodies produced by CHO cells with tetracycline-regulated expression of β (1,4)-N-acetylglucosaminyltransferase III (GnTIII), a glycosyltransferase catalyzing formation of bisecting GlcNAc, was reported to have improved ADCC activity (Umana et al., 1999, Nature Biotech. 17:176-180).

In some embodiments, the disclosure provides LILRB1×LILRB2 bispecific antibodies, anti-LILRB1 antibodies and anti-LILRB2 antibodies containing variations of the variable regions, the CDRs, or the heavy chain and light chain sequences as shown in Table 1, Table 2 and Table 3, respectively, wherein such variant polypeptides share at least 70%, at least 75%, at least 80%, at least 85%, at least 87%, at least 89%, at least 90%, at least 91%, at least 92%, at least 93%, at least 94%, at least 95%, at least 96%, at least 97%, at least 98%, or at least 99% amino acid sequence identity to any of the amino acid sequences disclosed in Table 1, 2, or 3. These amounts are not meant to be limiting and increments between the recited percentages are specifically envisioned as part of the disclosure.

The invention also encompasses fusion proteins comprising one or more components of the anti-LILRB1 antibodies or the anti-LILRB2 antibodies disclosed herein. In some embodiments, a fusion protein may be made that comprises all or a portion of an anti-LILRB1 antibody or anti-LILRB2 antibody of the invention linked to another polypeptide. In another embodiment, only the variable domains of the anti-LILRB1 antibody or anti-LILRB2 antibody are linked to the polypeptide. In another embodiment, the VH domain of an anti-LILRB1 antibody or anti-LILRB2 antibody is linked to a first polypeptide, while the VL domain of an anti-LILRB1 antibody or anti-LILRB2 antibody is linked to a second polypeptide that associates with the first polypeptide in a manner such that the VH and VL domains can interact with one another to form an antigen binding site. In another embodiment, the VH domain is separated from the VL domain by a linker such that the VH and VL domains can interact with one another. The VH-linker-VL antibody is then linked to the polypeptide of interest. In addition, fusion antibodies can be created in which two (or more) single-chain antibodies are linked to one another. This is useful if one wants to create a divalent or polyvalent antibody on a single polypeptide chain, or if one wants to create a bispecific antibody.

### Biological Activity of the LILRB1×LILRB2 Bispecific Antibodies, Anti-LILRB1 Antibodies, and Anti-LILRB2 Antibodies

In addition to binding an epitope on LILRB1, an epitope on LILRB2, or r binding to both, the LILRB1×LILRB2 bispecific antibodies, the anti-LILRB1 antibodies, and the anti-LILRB2 antibodies of the disclosure can mediate a biological activity. The disclosure includes an isolated antibody that specifically binds LILRB1, LILRB2 or both, and has or mediates at least one detectable activity selected from the following:
(i) binds specifically to human LILRB1, LILRB2, or both;
(ii) binds specifically to cynomolgus monkey LILRB1, LILRB2 or both;
(iii) reduces, inhibits or neutralizes interaction (e.g., binding) between HLA-G and soluble LILRB1, between HLA-G and soluble LILRB2, or both;
(iv) reduces, inhibits or neutralizes interaction (e.g., binding) between HLA-G and LILRB1 expressed on THP1 cells, between HLA-G and LILRB2 expressed on THP1 cells, or both;
(v) reduces, inhibits or neutralizes interaction between HLA-G and LILRB1 and/or LILRB2 expressed on human monocytes and increase cytokine release by the monocytes; and
(vi) reduces, inhibits or neutralizes interaction between HLA-G and LILRB1 and/or LILRB2 expressed on human leukocytes and increase cytokine release by these mixed leukocytes.

In some embodiments, the LILRB1×LILRB2 bispecific antibodies, the anti-LILRB1 antibodies, and the anti-LILRB2 antibodies do not bind to human LILRA1, A2, A3, A4, A5 and/or A6; and/or do not bind to human LILRB3, B4 and/or B5.

### Polynucleotides Encoding Soluble LILRB1 (e.g., Human, Cynomolgus) and HLA-G, and Methods of Manufacture

The disclosure also provides polynucleotides encoding any of the antibodies of the invention, including antibody portions and modified antibodies described herein. The invention also provides a method of making any of the antibodies and polynucleotides described herein. Polynucleotides can be made and the proteins expressed by procedures known in the art.

If desired, an anti-LILRB1 antibody or an anti-LILRB2 antibody (monoclonal or polyclonal) of interest may be sequenced and the polynucleotide sequence may then be cloned into a vector for expression or propagation. The sequence encoding the antibody of interest may be maintained in vector in a host cell and the host cell can then be expanded and frozen for future use. Production of recombinant monoclonal antibodies in cell culture can be carried out through cloning of antibody genes from B cells by means known in the art. See, e.g. Tiller et al., 2008, J. Immunol. Methods 329, 112; U.S. Pat. No. 7,314,622.

In some embodiments, provided herein is a polynucleotide comprising a sequence encoding one or both of the heavy chain or the light chain variable regions of an anti-LILRB1 antibody or an anti-LILRB2 antibody provided herein. The sequence encoding the antibody of interest may be maintained in a vector in a host cell and the host cell can then be expanded and frozen for future use. Vectors (including expression vectors) and host cells are further described herein.

It will be appreciated by those of ordinary skill in the art that, as a result of the degeneracy of the genetic code, there are many nucleotide sequences that encode a polypeptide as described herein. Some of these polynucleotides bear minimal homology to the nucleotide sequence of any native gene. Nonetheless, polynucleotides that vary due to differences in codon usage are specifically contemplated by the present invention. Further, alleles of the genes comprising the polynucleotide sequences provided herein are within the scope of the present invention. Alleles are endogenous genes that are altered as a result of one or more mutations, such as deletions, additions or substitutions of nucleotides. The resulting mRNA and protein may, but need not, have an altered structure or function. Alleles may be identified using standard techniques (such as hybridization, amplification or database sequence comparison).

In one embodiment, the VH and VL domains or full-length HC or LC, are encoded by separate polynucleotides. Alternatively, both VH and VL, or HC and LC, are encoded by a single polynucleotide.

Polynucleotides complementary to any such sequences are also encompassed by the present disclosure. Polynucleotides may be single-stranded (coding or antisense) or double-stranded, and may be DNA (genomic, cDNA or synthetic) or RNA molecules. RNA molecules include HnRNA molecules, which contain introns and correspond to a DNA molecule in a one-to-one manner, and mRNA molecules, which do not contain introns. Additional coding or non-coding sequences may, but need not, be present within a polynucleotide of the present disclosure, and a polynucleotide may, but need not, be linked to other molecules or support materials.

The polynucleotides of this invention can be obtained using chemical synthesis, recombinant methods, or PCR. Methods of chemical polynucleotide synthesis are well known in the art and need not be described in detail herein. One of skill in the art can use the sequences provided herein and a commercial DNA synthesizer to produce a desired DNA sequence.

For preparing polynucleotides using recombinant methods, a polynucleotide comprising a desired sequence can be inserted into a suitable vector, and the vector in turn can be introduced into a suitable host cell for replication and amplification, as further discussed herein. Polynucleotides may be inserted into host cells by any means known in the art. Cells are transformed by introducing an exogenous polynucleotide by direct uptake, endocytosis, transfection, F-mating or electroporation. Once introduced, the exogenous polynucleotide can be maintained within the cell as a non-integrated vector (such as a plasmid) or integrated into the host cell genome.

Suitable cloning vectors may be constructed according to standard techniques, or may be selected from a large number of cloning vectors available in the art. While the cloning vector selected may vary according to the host cell intended to be used, useful cloning vectors will generally have one or more features such as i) the ability to self-replicate, ii) a single target for a particular restriction endonuclease, or iii) may carry genes for a marker that can be used in selecting clones containing the vector. Suitable examples include plasmids and bacterial viruses, e.g., pUC18, pUC19, Bluescript (e.g., pBS SK+) and its derivatives, mp18, mp19, pBR322, pMB9, ColE1, pCR1, RP4, phage DNAs, and shuttle vectors such as pSA3 and pAT28. These and many other cloning vectors are available from commercial vendors such as BioRad, Strategene, and Invitrogen.

Expression vectors are further provided. Expression vectors generally are replicable polynucleotide constructs that contain a polynucleotide according to the invention. It is implied that an expression vector must be replicable in the host cells either as episomes or as an integral part of the chromosomal DNA. Suitable expression vectors include but are not limited to plasmids, viral vectors, including adenoviruses, adeno-associated viruses, retroviruses, cosmids, and expression vector(s) disclosed in PCT Publication No. WO 87/04462. Vector components may generally include, but are not limited to, one or more of the following: a signal sequence; an origin of replication; one or more marker genes; suitable transcriptional controlling elements (such as promoters, enhancers and terminator). For expression (i.e., translation), one or more translational controlling elements are also usually required, such as ribosome binding sites, translation initiation sites, and stop codons.

The vectors containing the polynucleotides of interest can be introduced into the host cell by any of a number of appropriate means, including electroporation, transfection employing calcium chloride, rubidium chloride, calcium phosphate, DEAE-dextran, or other substances; microprojectile bombardment; lipofection; and infection (e.g., where the vector is an infectious agent such as vaccinia virus). The choice of introducing vectors or polynucleotides will often depend on features of the host cell.

The invention also provides host cells comprising any of the polynucleotides described herein. Any host cells capable of over-expressing heterologous DNAs can be used for the purpose of isolating the genes encoding the antibody, polypeptide or protein of interest. Non-limiting examples of mammalian host cells include but not limited to COS, HeLa, and CHO cells. See also PCT Publication No. WO 87/04462. Suitable non-mammalian host cells include prokaryotes (such as E. coli or B. subtillis) and yeast (such as S. cerevisae, S. pombe; or K. lactis).

Additionally, any number of commercially and non-commercially available cell lines that express polypeptides or proteins may be utilized in accordance with the present invention. One skilled in the art will appreciate that different cell lines might have different nutrition requirements or might require different culture conditions for optimal growth and polypeptide or protein expression, and will be able to modify conditions as needed.

### Pharmaceutical Compositions

In another embodiment, the invention comprises pharmaceutical compositions.

A “pharmaceutical composition” refers to a mixture of an antibody the invention and one or excipient.

Pharmaceutical compositions of this invention may be in a variety of forms. These include, for example, liquid, semi-solid and solid dosage forms, such as liquid solutions (e.g., injectable and infusible solutions), dispersions or suspensions, and lyophilized powders. The form depends on the intended mode of administration and therapeutic application.

Other excipients and modes of administration known in the pharmaceutical art may also be used. Pharmaceutical compositions of the invention may be prepared by any of the well-known techniques of pharmacy, such as effective formulation and administration procedures. The above considerations in regard to effective formulations and administration procedures are well known in the art and are described in standard textbooks. Formulation of drugs is discussed in, for example, Hoover, John E., Remington's Pharmaceutical Sciences, Mack Publishing Co., Easton, Pennsylvania, 1975; Liberman et al., Eds., Pharmaceutical Dosage Forms, Marcel Decker, New York, N.Y., 1980; and Kibbe et al., Eds., Handbook of Pharmaceutical Excipients (3^{rd} Ed.), American Pharmaceutical Association, Washington, 1999.

Acceptable excipients are nontoxic to recipients at the dosages and concentrations employed, and may comprise buffers such as phosphate, citrate, and other organic acids; salts such as sodium chloride; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens, such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); or non-ionic surfactants such as TWEEN™, PLURONICS™ or polyethylene glycol (PEG).

### Therapeutic, Diagnostic, and Other Methods

The antibodies and the antibody conjugates of the present invention are useful in various applications including, but are not limited to, therapeutic treatment methods and diagnostic treatment methods.

In one aspect, the invention provides a method for treating cancer, especially solid tumors. In some embodiments, the cancer is mesothelioma, glioblastoma, renal cell carcinoma (RCC), non-small cell lung cancer (NSCLC), melanoma, biliary tract cancers (BTC), gastric cancer, head and neck squamous cell carcinoma (HNSCC), breast cancer, ovarian cancer, pancreatic cancer, cervical cancer, colorectal cancer (CRC) or esophageal cancer. In some embodiments, the cancer is NSCLC, RCC, urothelial carcinoma, bladder cancer, or melanoma. In some embodiments, the method of treating cancer in a subject comprises administering to the subject in need thereof an effective amount of a pharmaceutical composition comprising the LILRB1×LILRB2 bispecific antibody, the anti-LILRB1 antibody or the anti-LILRB2 antibody as described herein. In some embodiments, provided is a method of treating cancer, in a subject, comprising administering to the subject in need thereof an effective amount of a composition comprising an LILRB1×LILRB2 bispecific antibody, an anti-LILRB1 antibody or an anti-LILRB2 antibody described herein.

In another aspect, the invention further provides the LILRB1×LILRB2 bispecific antibody, the anti-LILRB1 antibody or the anti-LILRB2 antibody or pharmaceutical composition thereof as described herein for use in the described method of treating cancer. The invention also provides the use of the LILRB1×LILRB2 bispecific antibody, the anti-LILRB1 antibody or the anti-LILRB2 antibody as described herein in the manufacture of a medicament for treating cancer.

In another aspect, provided is a method of one or more of detecting, diagnosing, or monitoring cancer. For example, the LILRB1×LILRB2 bispecific antibody, the anti-LILRB1 antibody or the anti-LILRB2 antibody as described herein can be labeled with a detectable moiety such as an imaging agent and an enzyme-substrate label. The antibodies as described herein can also be used for in vivo in vitro and ex vivo diagnostic assays, prognosis assays such as in vivo imaging (e.g., PET or SPECT), or a staining reagent.

With respect to all methods described herein, reference to the LILRB1×LILRB2 bispecific antibody, the anti-LILRB1 antibody or the anti-LILRB2 antibody also includes pharmaceutical compositions comprising the LILRB1×LILRB2 bispecific antibody, the anti-LILRB1 antibody or the anti-LILRB2 antibody and one or more additional agents.

### Administration and Dosing

Typically, an antibody of the invention is administered in an amount effective to treat a condition as described herein. The antibodies the invention can be administered as an antibody per se, or alternatively, as a pharmaceutical composition containing the antibody.

The antibodies of the invention are administered by any suitable route in the form of a pharmaceutical composition adapted to such a route, and in a dose effective for the treatment intended.

In some embodiments, the antibodies may be administered parenterally, for example directly into the bloodstream, into muscle, or into an internal organ. Suitable means for parenteral administration include intravenous, intraarterial, intraperitoneal, intrathecal, intraventricular, intraurethral, intrasternal, intracranial, intramuscular and subcutaneous. In some embodiments, the antibodies may be administered intravenously or subcutaneously. Suitable devices for parenteral administration include needle (including microneedle) injectors, needle-free injectors, and infusion techniques.

In another embodiment, the compounds of the invention may also be administered topically to the skin or mucosa, that is, dermally or transdermally. In another embodiment, the compounds of the invention can also be administered intranasally or by inhalation. In another embodiment, the compounds of the invention may be administered rectally or vaginally. In another embodiment, the compounds of the invention may also be administered directly to the eye or ear.

The dosage regimen for the antibodies of the invention or compositions containing said antibodies is based on a variety of factors, including the type, age, weight, sex and medical condition of the subject; the severity of the condition; the route of administration; and the activity of the particular antibody employed. Thus, the dosage regimen may vary widely. In one embodiment, the total daily dose of an antibody of the invention is typically from about 0.01 to about 100 mg/kg (i.e., mg antibody of the invention per kg body weight) for the treatment of the indicated conditions discussed herein. In another embodiment, total daily dose of the antibody of the invention is from about 0.1 to about 50 mg/kg, and in another embodiment, from about 0.5 to about 30 mg/kg.

### Co-Administration

The antibodies of the invention can be used alone, or in combination with one or more other therapeutic agents. The invention provides any of the uses, methods or compositions as defined herein wherein an antibody of the invention is used in combination with one or more other therapeutic agent discussed herein. In some embodiments, the other therapeutic agent is an anti-PD-1 antibody. (Siu et. al. Clin Cancer Res (2022) 28 (1): 57-70). In some embodiments, the anti-PD-1 antibody is pembrolizumab, nivolumab or sasanlimab.

The administration of two or more agents “in combination” means that all of the agents are administered closely enough in time to affect treatment of the subject. The two or more agents may be administered simultaneously or sequentially. Additionally, simultaneous administration may be carried out by mixing the agents prior to administration or by administering the agents at the same point in time but as separate dosage forms at the same or different site of administration.

### Kits

Another aspect of the invention provides kits comprising the antibody of the invention or pharmaceutical compositions comprising the antibody. A kit may include, in addition to the antibody of the invention or pharmaceutical composition thereof, diagnostic or therapeutic agents. A kit may also include instructions for use in a diagnostic or therapeutic method. In some embodiments, the kit includes the antibody or a pharmaceutical composition thereof and a diagnostic agent. In other embodiments, the kit includes the antibody or a pharmaceutical composition thereof and one or more therapeutic agents.

In yet another embodiment, the invention comprises kits that are suitable for use in performing the methods of treatment described herein. In one embodiment, the kit contains a first dosage form comprising one or more of the antibodies of the invention in quantities sufficient to carry out the methods of the invention. In another embodiment, the kit comprises one or more antibodies of the invention in quantities sufficient to carry out the methods of the invention and at least a first container for a first dosage and a second container for a second dosage.

### Biological Deposits

Representative materials of the present invention were deposited in the American Type Culture Collection (ATCC) on Jan. 25, 2024. Plasmid having ATCC Accession No. PTA-127715 contains a nucleic acid sequence encoding the amino acid sequence of the B1 arm VH of bispecific antibody BsAb-1882. Plasmid having ATCC Accession No. PTA-127716 contains a nucleic acid sequence encoding the amino acid sequence of the B1 arm VL of bispecific antibody BsAb-1882. Plasmid having ATCC Accession No. PTA-127717 contains a nucleic acid sequence encoding the amino acid sequence of the B2 arm VH of bispecific antibody BsAb-1882. Plasmid having ATCC Accession No. PTA-127718 contains a nucleic acid sequence encoding the amino acid sequence of the B2 arm VL of bispecific antibody BsAb-1882.

The deposits were made under the provisions of the Budapest Treaty on the International Recognition of the Deposit of Microorganisms for the Purpose of Patent Procedure and Regulations thereunder (Budapest Treaty). This assures maintenance of a viable culture of the deposit for 30 years from the date of deposit. The deposit will be made available by ATCC under the terms of the Budapest Treaty, and subject to an agreement between Pfizer Inc. and ATCC, which assures permanent and unrestricted availability of the progeny of the culture of the deposit to the public upon issuance of the pertinent U.S. patent or upon laying open to the public of any U.S. or foreign patent application, whichever comes first, and assures availability of the progeny to one determined by the U.S. Commissioner of Patents and Trademarks to be entitled thereto according to 35 U.S.C. Section 122 and the Commissioner's rules pursuant thereto (including 37 C.F.R. Section 1.14 with particular reference to 886 OG 638).

The assignee of the present application has agreed that if a culture of the materials on deposit should die or be lost or destroyed when cultivated under suitable conditions; the materials will be promptly replaced on notification with another of the same. Availability of the deposited material is not to be construed as a license to practice the invention in contravention of the rights granted under the authority of any government in accordance with its patent laws.

### Sequences

The amino acid sequences and nucleotide sequences referred to In Tables 1-3 are described in below Table 4. Kabat and Chothia VH CDR1 and VH CDR2 are marked using underline and bold/italic respectively in the heavy chain and VH sequences.

TABLE 4
Sequences

| Seq ID | | |
|---|---|---|
| NO. | Description | Sequence |
| 1 | BsAb-1882 B1 arm | SYELTQPPSV SVSPGQTASI TCGGNNIGSK |
| | Light chain | GVHWYQQKPG QSPVLVISYD SDRPSGIPER |
| | VL-CL-hinge-Fc | FSGSNSGNTA TLTISGTQAM DEADYYCQVW |
| | | DSSTRVFGTG TKVTVLGQPK AAPSVTLFPP |
| | | SSEELQANKA TLVCLISDFY PGAVTVAWKA |
| | | DSSPVKAGVE TTTPSKQSNN KYAASSYLSL |
| | | TPEQWKSHRS YSCQVTHEGS TVEKTVAPTE |
| | | CSDKTHTCPP CPAPEAAGAP SVFLFPPKPK |
| | | DTLMISRTPE VTCVVVDVSH EDPEVKFNWY |
| | | VDGVEVHNAK TKPREEQYNS TYRVVSVLTV |
| | | LHQDWLNGKE YKCKVSNKAL PAPIEKTISK |
| | | AKGQPREPQV YTLPPCREEM TKNQVSLSCA |
| | | VKGFYPSDIA VEWESNGQPE NNYKTTPPVL |
| | | DSDGSFFLVS KLTVDKSRWQ QGNVFSCSVM |
| | | HEALHNHYTQ KSLSLSPGK |
| 2 | BsAb-1882 B1 arm, | GGNNIGSK GVH |
| | Ab_B1-1704, | |
| | Light chain VL CDR1 | |
| 3 | BsAb-1882 B1 arm, | YD SDRPS |
| | Ab_B1-1704, | |
| | Light chain VL CDR2 | |
| 4 | BsAb-1882 B1 arm, | QVW DSSTRV |
| | Ab_B1-1704, | |
| | Light chain VL CDR3 | |
| 5 | BsAb-1882 B1 arm, | GQPK AAPSVTLFPP SSEELQANKA TLVCLISDFY |
| | Ab_B1-1704, | PGAVTVAWKA DSSPVKAGVE TTTPSKQSNN |
| | Light chain CL | KYAASSYLSL TPEQWKSHRS YSCQVTHEGS |
| | | TVEKTVAPTE CS |
| 6 | BsAb-1882 B1 arm; | SYELTQPPSV SVSPGQTASI TCGGNNIGSK |
| | Ab_B1-1704, | GVHWYQQKPG QSPVLVISYD SDRPSGIPER |
| | Light chain VL | FSGSNSGNTA TLTISGTQAM DEADYYCQVW |
| | | DSSTRVFGTG TKVTVL |
| 7 | BsAb-1882 B1 arm | APEAAGAP SVFLFPPKPK DTLMISRTPE |
| | Light chain Fc | VTCVVVDVSH EDPEVKFNWY VDGVEVHNAK |
| | | TKPREEQYNS TYRVVSVLTV LHQDWLNGKE |
| | | YKCKVSNKAL PAPIEKTISK AKGQPREPQV |
| | | YTLPPCREEM TKNQVSLSCA VKGFYPSDIA |
| | | VEWESNGQPE NNYKTTPPVL DSDGSFFLVS |
| | | KLTVDKSRWQ QGNVFSCSVM HEALHNHYTQ |
| | | KSLSLSPG(K) |
| 8 | BsAb-1882 B1 arm | EVQLVESGGG LVQPGGSLRL SCAASGFTFS |
| | heavy chain, | SYAMHWVRQA PGKGLEWVAV ISYDGSNKYY |
| | VH-CH1; | ADSVKGRFTI SRDNAKNSLY LQMNSLRAED |
| | Ab_B1-1704, | TAVYYCARDK GIGTSPLDYW GQGTLVTVSS |
| | heavy chain VH- | ASTKGPSVFP LAPSSKSTSG GTAALGCLVK |
| | CH1 | DYFPEPVTVS WNSGALTSGV HTFPAVLQSS |
| | | GLYSLSSVVT VPSSSLGTQT YICNVNHKPS |
| | | NTKVDKKVEP KSC |
| 9 | BsAb-1882 B1 arm; | SYAMH |
| | Ab_B1-1704, | |
| | VH CDR1 (Kabat) | |
| 10 | BsAb-1882 B1 arm, | GFTFSSY |
| | Ab_B1-1704, | |
| | VH CDR1 (chothia) | |
| 11 | BsAb-1882 B1 arm, | GFTFSSYAMH |
| | Ab_B1-1704, | |
| | VH CDR1 (extended) | |
| 12 | BsAb-1882 B1 arm, | V ISYDGSNKYY ADSVKG |
| | Ab_B1-1704, | |
| | VH CDR2 (kabat) | |
| 13 | BsAb-1882 B1 arm | SYDGSN |
| | VH CDR2 (Chothia) | |
| 14 | BsAb-1882 B1 arm, | DK GIGTSPLDY |
| | Ab_B1-1704, | |
| | VH CDR3 | |
| 15 | BsAb-1882 B1 arm, | ASTKGPSVFP LAPSSKSTSG GTAALGCLVK |
| | Ab_B1-1704, | DYFPEPVTVS WNSGALTSGV HTFPAVLQSS |
| | CH1 | GLYSLSSVVT VPSSSLGTQT YICNVNHKPS |
| | | NTKVDKKVEP KSC |
| 16 | BsAb-1882 B1 arm | EVQLVESGGG LVQPGGSLRL SCAASGFTFS |
| | Ab_B1-1704 | SYAMHWVRQA PGKGLEWVAV ISYDGSNKYY |
| | VH | ADSVKGRFTI SRDNAKNSLY LQMNSLRAED |
| | | TAVYYCARDK GIGTSPLDYW GQGTLVTVSS |
| 17 | BsAb-1882 B2 arm | QSVLTQPPSA SGTPGQRVTI SCTGSSSNIG |
| | BsAb-1880 B2 arm | SNTVNWYQQL PGTAPKLLIY SDNQRPSGVP |
| | Ab_B2-1825 | DRFSGSTSGT SASLAISGLR SEDEADYFCA |
| | Light chain | TWEDVLAGYV VFGGGTKLTV LGQPKAAPSV |
| | | TLFPPSSEEL QANKATLVCL ISDFYPGAVT |
| | | VAWKADSSPV KAGVETTTPS KQSNNKYAAS |
| | | SYLSLTPEQW KSHRSYSCQV THEGSTVEKT |
| | | VAPTECS |
| 18 | BsAb-1882, B2 arm | TGSSSNIG SNTVN |
| | Ab_B2-1825 | |
| | VL CDR1 | |
| 19 | BsAb-1882, B2 arm | SDNQRPS |
| | Ab_B2-1825 | |
| | VL CDR2 | |
| 20 | BsAb-1882 B2 arm, | ATWEDVLAGYVV |
| | Ab_B2-1825, | |
| | VL CDR3 | |
| 21 | BsAb-1882 B2 arm, | GQPKAAPSV TLFPPSSEEL QANKATLVCL |
| | Ab_B2-1825, | ISDFYPGAVT VAWKADSSPV KAGVETTTPS |
| | CL | KQSNNKYAAS SYLSLTPEQW KSHRSYSCQV |
| | | THEGSTVEKT VAPTECS |
| 22 | BsAb-1882 B2 arm, | QSVLTQPPSA SGTPGQRVTI SCTGSSSNIG |
| | Ab_B2-1825, | SNTVNWYQQL PGTAPKLLIY SDNQRPSGVP |
| | VL | DRFSGSTSGT SASLAISGLR SEDEADYFCA |
| | | TWEDVLAGYV VFGGGTKLTV L |
| 23 | BsAb-1882 B2 arm | EVQLLESGGG LVQPGGSLRL SCAASGFTFS |
| | Heavy chain | SYAMSWVRQA PGKGLEWVSA ISGSGGSTYY |
| | | ADSVKGRFTI SRDNSKNTLY LQMNSLRAED |
| | | TAVYYCWRWL NLGDAFDIWG QGTMVTVSSA |
| | | STKGPSVFPL APSSKSTSGG TAALGCLVKD |
| | | YFPEPVTVSW NSGALTSGVH TFPAVLQSSG |
| | | LYSLSSVVTV PSSSLGTQTY ICNVNHKPSN |
| | | TKVDKKVEPK SCDKTHTCPP CPAPEAAGAP |
| | | SVFLFPPKPK DTLMISRTPE VTCVVVDVSH |
| | | EDPEVKFNWY VDGVEVHNAK TKPREEQYNS |
| | | TYRVVSVLTV LHQDWLNGKE YKCKVSNKAL |
| | | PAPIEKTISK AKGQPREPQV CTLPPSREEM |
| | | TKNQVSLWCL VKGFYPSDIA VEWESNGQPE |
| | | NNYKTTPPVL DSDGSFFLYS KLTVDKSRWQ |
| | | QGNVFSCSVM HEALHNHYTQ |
| | | KSLSLSPGK |
| 24 | BsAb-1882, B2 arm, | SYAMS |
| | Ab_B2-1825, | |
| | VH CDR1 (kabat) | |
| 25 | BsAb-1882 B2 arm, | GFTFSSY |
| | Ab_B2-1825, | |
| | VH CDR1 (Chothia) | |
| 26 | BsAb-1882 B2 arm, | GFTFSSYAMS |
| | Ab_B2-1825, | |
| | VH CDR1 (extended) | |
| 27 | BsAb-1882 B2 arm, | A ISGSGGSTYY ADSVKG |
| | Ab_B2-1825, | |
| | VH CDR2 (kabat) | |
| 28 | BsAb-1882 B2 arm, | SGSGGS |
| | Ab_B2-1825, | |
| | VH CDR2 (Chothia) | |
| 29 | BsAb-1882 B2 arm, | WLNLGDAFDI |
| | Ab_B2-1825, | |
| | VH CDR3 | |
| 30 | BsAb-1882 | A STKGPSVFPL APSSKSTSGG TAALGCLVKD |
| | B2 arm | YFPEPVTVSW NSGALTSGVH TFPAVLQSSG |
| | Constant region | LYSLSSVVTV PSSSLGTQTY ICNVNHKPSN |
| | | TKVDKKVEPK SCDKTHTCPP CPAPEAAGAP |
| | | SVFLFPPKPK DTLMISRTPE VTCVVVDVSH |
| | | EDPEVKFNWY VDGVEVHNAK TKPREEQYNS |
| | | TYRVVSVLTV LHQDWLNGKE YKCKVSNKAL |
| | | PAPIEKTISK AKGQPREPQV CTLPPSREEM |
| | | TKNQVSLWCL VKGFYPSDIA VEWESNGQPE |
| | | NNYKTTPPVL DSDGSFFLYS KLTVDKSRWQ |
| | | QGNVFSCSVM HEALHNHYTQ |
| | | KSLSLSPG(K) |
| 31 | BsAb-1882, B2 arm | EVQLLESGGG LVQPGGSLRL SCAASGFTFS |
| | Ab_B2-1825, | SYAMSWVRQA PGKGLEWVSA ISGSGGSTYY |
| | VH | ADSVKGRFTI SRDNSKNTLY LQMNSLRAED |
| | | TAVYYCWRWL NLGDAFDIWG QGTMVTVSS |
| 32 | BsAb-1882 | A STKGPSVFPL APSSKSTSGG TAALGCLVKD |
| | CH1 | YFPEPVTVSW NSGALTSGVH TFPAVLQSSG |
| | | LYSLSSVVTV PSSSLGTQTY |
| | | TKVDKKVEPK SC |
| 33 | BsAb-1882 B2 arm | DKTHTCPPCP |
| | heavy chain | |
| | hinge | |
| | BsAb-1882 B1 arm | |
| | light chain hinge | |
| 34 | BsAb-1882 B2 arm | APEAAGAP SVFLFPPKPK DTLMISRTPE |
| | Heavy chain | VTCVVVDVSH EDPEVKFNWY VDGVEVHNAK |
| | Fc | TKPREEQYNS TYRVVSVLTV LHQDWLNGKE |
| | | YKCKVSNKAL PAPIEKTISK AKGQPREPQV |
| | | CTLPPSREEM TKNQVSLWCL VKGFYPSDIA |
| | | VEWESNGQPE NNYKTTPPVL DSDGSFFLYS |
| | | KLTVDKSRWQ QGNVFSCSVM HEALHNHYTQ |
| | | KSLSLSPG(K) |
| 35 | BsAb-1882 B1 arm | SYELTQPPSV SVSPGQTASI TCGGNNIGSK |
| | Light chain without C | GVHWYQQKPG QSPVLVISYD SDRPSGIPER |
| | terminal lysine | FSGSNSGNTA TLTISGTQAM DEADYYCQVW |
| | | DSSTRVFGTG TKVTVLGQPK AAPSVTLFPP |
| | | SSEELQANKA TLVCLISDFY PGAVTVAWKA |
| | | DSSPVKAGVE TTTPSKQSNN KYAASSYLSL |
| | | TPEQWKSHRS YSCQVTHEGS TVEKTVAPTE |
| | | CSDKTHTCPP CPAPEAAGAP SVFLFPPKPK |
| | | DTLMISRTPE VTCVVVDVSH EDPEVKFNWY |
| | | VDGVEVHNAK TKPREEQYNS TYRVVSVLTV |
| | | LHQDWLNGKE YKCKVSNKAL PAPIEKTISK |
| | | AKGQPREPQV YTLPPCREEM TKNQVSLSCA |
| | | VKGFYPSDIA VEWESNGQPE NNYKTTPPVL |
| | | DSDGSFFLVS KLTVDKSRWQ QGNVFSCSVM |
| | | HEALHNHYTQ KSLSLSPG |
| 36 | BsAb-1882 B2 arm | EVQLLESGGG LVQPGGSLRL SCAASGFTFS |
| | Heavy chain without | SYAMSWVRQA PGKGLEWVSA ISGSGGSTYY |
| | C terminal lysine | ADSVKGRFTI SRDNSKNTLY LQMNSLRAED |
| | | TAVYYCWRWL NLGDAFDIWG QGTMVTVSSA |
| | | STKGPSVFPL APSSKSTSGG TAALGCLVKD |
| | | YFPEPVTVSW NSGALTSGVH TFPAVLQSSG |
| | | LYSLSSVVTV PSSSLGTQTY ICNVNHKPSN |
| | | TKVDKKVEPK SCDKTHTCPP CPAPEAAGAP |
| | | SVFLFPPKPK DTLMISRTPE VTCVVVDVSH |
| | | EDPEVKFNWY VDGVEVHNAK TKPREEQYNS |
| | | TYRVVSVLTV LHQDWLNGKE YKCKVSNKAL |
| | | PAPIEKTISK AKGQPREPQV CTLPPSREEM |
| | | TKNQVSLWCL VKGFYPSDIA VEWESNGQPE |
| | | NNYKTTPPVL DSDGSFFLYS KLTVDKSRWQ |
| | | QGNVFSCSVM HEALHNHYTQ |
| | | KSLSLSPG |
| 37 | Ab_B1-1704 | SYELTQPPSV SVSPGQTASI TCGGNNIGSK |
| | Light chain | GVHWYQQKPG QSPVLVISYD SDRPSGIPER |
| | | FSGSNSGNTA TLTISGTQAM DEADYYCQVW |
| | | DSSTRVFGTG TKVTVLGQPK AAPSVTLFPP |
| | | SSEELQANKA TLVCLISDFY PGAVTVAWKA |
| | | DSSPVKAGVE TTTPSKQSNN KYAASSYLSL |
| | | TPEQWKSHRS YSCQVTHEGS TVEKTVAPTE CS |
| 38 | Ab_B1- 1704 | EVQLVESGGG LVQPGGSLRL SCAASGFTFS |
| | Heavy chain | SYAMHWVRQA PGKGLEWVAV ISYDGSNKYY |
| | | ADSVKGRFTI SRDNAKNSLY LQMNSLRAED |
| | | TAVYYCARDK GIGTSPLDYW GQGTLVTVSS |
| | | ASTKGPSVFP LAPSSKSTSG GTAALGCLVK |
| | | DYFPEPVTVS WNSGALTSGV HTFPAVLQSS |
| | | GLYSLSSVVT VPSSSLGTQT YICNVNHKPS |
| | | NTKVDKKVEP KSCDKTHTCP PCPAPEAAGA |
| | | PSVFLFPPKP KDTLMISRTP EVTCVVVDVS |
| | | HEDPEVKFNW YVDGVEVHNA KTKPREEQYN |
| | | STYRVVSVLT VLHQDWLNGK EYKCKVSNKA |
| | | LPAPIEKTIS KAKGQPREPQ VYTLPPSREE |
| | | MTKNQVSLTC LVKGFYPSDI AVEWESNGQP |
| | | ENNYKTTPPV LDSDGSFFLY SKLTVDKSRW |
| | | QQGNVFSCSV MHEALHNHYT QKSLSLSPGK |
| 39 | Ab_B2-1825 | EVQLLESGGG LVQPGGSLRL SCAASGFTFS |
| | Heavy chain | SYAMSWVRQA PGKGLEWVSA ISGSGGSTYY |
| | | ADSVKGRFTI SRDNSKNTLY LQMNSLRAED |
| | | TAVYYCWRWL NLGDAFDIWG QGTMVTVSSA |
| | | STKGPSVFPL APSSKSTSGG TAALGCLVKD |
| | | YFPEPVTVSW NSGALTSGVH TFPAVLQSSG |
| | | LYSLSSVVTV PSSSLGTQTY ICNVNHKPSN |
| | | TKVDKKVEPK SCDKTHTCPP CPAPEAAGAP |
| | | SVFLFPPKPK DTLMISRTPE VTCVVVDVSH |
| | | EDPEVKFNWY VDGVEVHNAK TKPREEQYNS |
| | | TYRVVSVLTV LHQDWLNGKE YKCKVSNKAL |
| | | PAPIEKTISK AKGQPREPQV YTLPPSREEM |
| | | TKNQVSLTCL VKGFYPSDIA VEWESNGQPE |
| | | NNYKTTPPVL DSDGSFFLYS KLTVDKSRWQ |
| | | QGNVFSCSVM HEALHNHYTQ KSLSLSPGK |
| 40 | BsAb-1880 | SYELTQPPSV SVSPGQTASI TCGGNNIGSK |
| | B1 arm | GVHWYQQKPG QSPVLVISYD SDRPSGIPER |
| | Light chain | FSGSNSGNTA TLTISGTQAM DEADYYCQVW |
| | | DSSTRVFGTG TKVTVLGQPK AAPSVTLFPP |
| | | SSEELQANKA TLVCLISDFY PGAVTVAWKA |
| | | DSSPVKAGVE TTTPSKQSNN KYAASSYLSL |
| | | TPEQWKSHRS YSCQVTHEGS TVEKTVAPTE CS |
| 41 | BsAb-1880 | EVQLVESGGG LVQPGGSLRL SCAASGFTFS |
| | B1 arm | SYAMHWVRQA PGKGLEWVAV ISYDGSNKYY |
| | Heavy chain | ADSVKGRFTI SRDNAKNSLY LQMNSLRAED |
| | | TAVYYCARDK GIGTSPLDYW GQGTLVTVSS |
| | | ASTKGPSVFP LAPSSKSTSG GTAALGCLVK |
| | | DYFPEPVTVS WNSGALTSGV HTFPAVLQSS |
| | | GLYSLSSVVT VPSSSLGTQT YICNVNHKPS |
| | | NTKVDKKVEP KSCRKTHTCP PCPAPEAAGA |
| | | PSVFLFPPKP KDTLMISRTP EVTCVVVDVS |
| | | HEDPEVKFNW YVDGVEVHNA KTKPREEQYN |
| | | STYRVVSVLT VLHQDWLNGK EYKCKVSNKA |
| | | LPAPIEKTIS KAKGQPREPQ VYTLPPSREE |
| | | MTKNQVSLTC LVKGFYPSDI AVEWESNGQP |
| | | ENNYKTTPPV LDSDGSFFLY SRLTVDKSRW |
| | | QQGNVFSCSV MHEALHNHYT QKSLSLSPGK |
| 42 | BsAb-1880 | EVQLLESGGG LVQPGGSLRL SCAASGFTFS |
| | B2 arm | SYAMSWVRQA PGKGLEWVSA ISGSGGSTYY |
| | Heavy chain | ADSVKGRFTI SRDNSKNTLY LQMNSLRAED |
| | | TAVYYCWRWL NLGDAFDIWG QGTMVTVSSA |
| | | STKGPSVFPL APSSKSTSGG TAALGCLVKD |
| | | YFPEPVTVSW NSGALTSGVH TFPAVLQSSG |
| | | LYSLSSVVTV PSSSLGTQTY ICNVNHKPSN |
| | | TKVDKKVEPK SCEKTHTCPP CPAPEAAGAP |
| | | SVFLFPPKPK DTLMISRTPE VTCVVVDVSH |
| | | EDPEVKFNWY VDGVEVHNAK TKPREEQYNS |
| | | TYRVVSVLTV LHQDWLNGKE YKCKVSNKAL |
| | | PAPIEKTISK AKGQPREPQV YTLPPSREEM |
| | | TKNQVSLTCE VKGFYPSDIA VEWESNGQPE |
| | | NNYKTTPPVL DSDGSFFLYS KLTVDKSRWQ |
| | | QGNVFSCSVM HEALHNHYTQ KSLSLSPGK |
| 43 | Human wildtype | ASTKGPSVFP LAPSSKSTSG GTAALGCLVK |
| | IgG1 heavy chain | DYFPEPVTVS WNSGALTSGV HTFPAVLQSS |
| | constant region | GLYSLSSVVT VPSSSLGTQT YICNVNHKPS |
| | | NTKVDKKVEP KSCDKTHTCP PCPAPELLGG |
| | | PSVFLFPPKP KDTLMISRTP EVTCVVVDVS |
| | | HEDPEVKFNW YVDGVEVHNA KTKPREEQYN |
| | | STYRVVSVLT VLHQDWLNGK EYKCKVSNKA |
| | | LPAPIEKTIS KAKGQPREPQ VYTLPPSRDE |
| | | LTKNQVSLTC LVKGFYPSDI AVEWESNGQP |
| | | ENNYKTTPPV LDSDGSFFLY SKLTVDKSRW |
| | | QQGNVFSCSV MHEALHNHYT QKSLSLSPGK |
| 44 | Human wildtype | ASTKGPSVFP LAPCSRSTSE STAALGCLVK |
| | IgG2 heavy chain | DYFPEPVTVS WNSGALTSGV HTFPAVLQSS |
| | constant region | GLYSLSSVVT VPSSNFGTQT YTCNVDHKPS |
| | | NTKVDKTVER KCCVECPPCP APPVAGPSVF |
| | | LFPPKPKDTL MISRTPEVTC VVVDVSHEDP |
| | | EVQFNWYVDG VEVHNAKTKP REEQFNSTFR |
| | | VVSVLTVVHQ DWLNGKEYKC KVSNKGLPAP |
| | | IEKTISKTKG QPREPQVYTL PPSREEMTKN |
| | | QVSLTCLVKG FYPSDIAVEW ESNGQPENNY |
| | | KTTPPMLDSD GSFFLYSKLT VDKSRWQQGN |
| | | VFSCSVMHEA LHNHYTQKSL SLSPGK |
| 45 | Human wildtype | ASTKGPSVFP LAPCSRSTSG GTAALGCLVK |
| | IgG3 heavy chain | DYFPEPVTVS WNSGALTSGV HTFPAVLQSS |
| | constant region | GLYSLSSVVT VPSSSLGTQT YTCNVNHKPS |
| | | NTKVDKRVEL KTPLGDTTHT CPRCPEPKSC |
| | | DTPPPCPRCP EPKSCDTPPP CPRCPEPKSC |
| | | DTPPPCPRCP APELLGGPSV FLFPPKPKDT |
| | | LMISRTPEVT CVVVDVSHED PEVQFKWYVD |
| | | GVEVHNAKTK PREEQYNSTF RVVSVLTVLH |
| | | QDWLNGKEYK CKVSNKALPA PIEKTISKTK |
| | | GQPREPQVYT LPPSREEMTK NQVSLTCLVK |
| | | GFYPSDIAVE WESSGQPENN YNTTPPMLDS |
| | | DGSFFLYSKL TVDKSRWQQG NIFSCSVMHE |
| | | ALHNRFTQKS LSLSPGK |
| 46 | Human wildtype | ASTKGPSVFP LAPCSRSTSE STAALGCLVK |
| | IgG4 heavy chain | DYFPEPVTVS WNSGALTSGV HTFPAVLQSS |
| | constant region | GLYSLSSVVT VPSSSLGTKT YTCNVDHKPS |
| | | NTKVDKRVES KYGPPCPSCP APEFLGGPSV |
| | | FLFPPKPKDT LMISRTPEVT CVVVDVSQED |
| | | PEVQFNWYVD GVEVHNAKTK PREEQFNSTY |
| | | RVVSVLTVLH QDWLNGKEYK CKVSNKGLPS |
| | | SIEKTISKAK GQPREPQVYT LPPSQEEMTK |
| | | NQVSLTCLVK GFYPSDIAVE WESNGQPENN |
| | | YKTTPPVLDS DGSFFLYSRL TVDKSRWQEG |
| | | NVFSCSVMHE ALHNHYTQKS LSLSLGK |
| 47 | Human lambda light | GQPKANPTVT LFPPSSEELQ ANKATLVCLI |
| | chain constant | SDFYPGAVTV AWKADGSPVK AGVETTKPSK |
| | region 1 | QSNNKYAASS YLSLTPEQWK SHRSYSCQVT |
| | | HEGSTVEKTV APTECS |
| 48 | Human lambda light | GQPKAAPSVT LFPPSSEELQ ANKATLVCLI |
| | chain constant | SDFYPGAVTV AWKADSSPVK AGVETTTPSK |
| | region 2 | QSNNKYAASS YLSLTPEQWK SHRSYSCQVT |
| | | HEGSTVEKTV APTECS |
| 49 | Human kappa light | GQPKAAPSVT LFPPSSEELQ ANKATLVCLI |
| | chain constant | SDFYPGAVTV AWKADSSPVK AGVETTTPSK |
| | region | QSNNKYAASS YLSLTPEQWK SHRSYSCQVT |
| | | HEGSTVEKTV APTECS |

The following examples of specific aspects for carrying out the present invention are offered for illustrative purposes only, and are not intended to limit the scope of the present invention in any way.

The foregoing description and following Examples detail certain specific embodiments of the disclosure and describes the best mode contemplated by the inventors. It will be appreciated, however, that no matter how detailed the foregoing may appear in text, the disclosure may be practiced in many ways and the disclosure should be construed in accordance with the appended claims and any equivalents thereof.

Although the disclosed teachings have been described with reference to various applications, methods, kits, and compositions, it will be appreciated that various changes and modifications can be made without departing from the teachings herein and the claimed disclosure below. The following examples are provided to better illustrate the disclosed teachings and are not intended to limit the scope of the teachings presented herein. While the present teachings have been described in terms of these exemplary embodiments, the skilled artisan will readily understand that numerous variations and modifications of these exemplary embodiments are possible without undue experimentation. All such variations and modifications are within the scope of the current teachings.

### Examples

### Example 1: Binding Affinity and Specificity of AB_B1-1704 and AB_B2-1825 Assessed by Surface Plasmon Resonance (SPR)

Anti-LILRB1 antibody AB_B1-1704 and anti-LILRB2 antibody AB_B2-1825 were tested for their binding affinities to huLILRB1, huLILIB2, and huLILRA1, A2 and A5.

Biacore kinetic assays were conducted at 37° C. with a collection rate of 10 Hz using the Biacore 8K and 8K+ instruments (Cytiva). Antibodies AB-B2-1825 or AB_B1-1704 were captured by an anti-human IgG (Fc specific) antibody (BR-1008-39, Cytiva) covalently coupled onto a CM5 sensor chip (29-1496-03, Cytiva) according to the manufacturer's instructions. The final capture levels of the antibodies ranged from 20 resonance units (RU) to 60 RU. HBS-EP+pH 7.4 (10 mM HEPES pH 7.4, 150 mM NaCl, 3 mM EDTA, 0.05% Tween 20) was used as sample and running buffer. Flow cell 1 was used as a reference flow cell. Two-fold serial dilutions of human LILRB2 or LILRB1 (R&D Systems, Catalog number: 8429-T4) were prepared with concentrations from 45 nM to 1.667 nM for LILRB2; 405 nM to 15 nM for LILRB1. Dilutions were prepared in duplicate and injected over all flow cells for 60 s at flow rate of 50 μl per minute. Dissociation was monitored for 600 s and the surface was regenerated with two 30 s injections of 3M MgCl₂ at flow rate of 50 μl per minute. The resulting data was double referenced (Myszka, D. G. Improving biosensor analysis. J. Mol. Recognit. 12:279-284; (1999). Rate constants and affinities were determined by fitting the sensorgram data to a Langmuir 1:1 model using Biacore Insight Evaluation software version 3.0.12 (Cytiva).

The results are described in Table 5. As used therein, k_{d} refers to association constant, k_{d} refers to dissociation constant and K_{D} refers to equilibrium dissociation constant.

TABLE 5
Binding Affinities of Antibodies to huLILRB1 and huLILRB2

| Analyte | Antibody | ka (1/Ms) | kd (1/s) | t½ (s) | K_{D} (nM) |
|---|---|---|---|---|---|
| huLILRB1 | Ab_B1- | 5.39E+05 | 1.40E−02 | 4.95E+01 | 26.01 ± 0.29 |
| | 1704 | | | | |
| huLILRB2 | Ab_B2- | 8.10E+05 | 2.76E−04 | 3.25E+03 | 0.59 ± 0.3 |
| | 1825 | | | | |

The binding of antibodies Ab_B1-1704 and Ab_B2-1825 to LILRA family members (A1, A2 and A5) were also examined and no binding was observed for either antibody.

This Example demonstrated that Anti-LILRB1 antibody Ab_B1-1704 binds strongly to huLILRB1, and Anti-LILRB2 antibody Ab_B2-1825 binds strongly to huLILRB2, and both antibodies are highly selective in their binding to the LILR family.

### Example 2: Inhibition of the Interaction HLA-G Tetramer and LILRB1 or LILRB2 Expressed on THP1 Cells by AB_B1-1704 and AB_B2-1825

Anti-huLILRB1 antibody AB_B1-1704 and anti-huLILRB2 antibody AB_B2-1825 were tested in cell-based ligand competition assays for their inhibition of the interaction of recombinant HLA-G tetramer with either LILRB1 or LILRB2 expressed on THP1 cells.

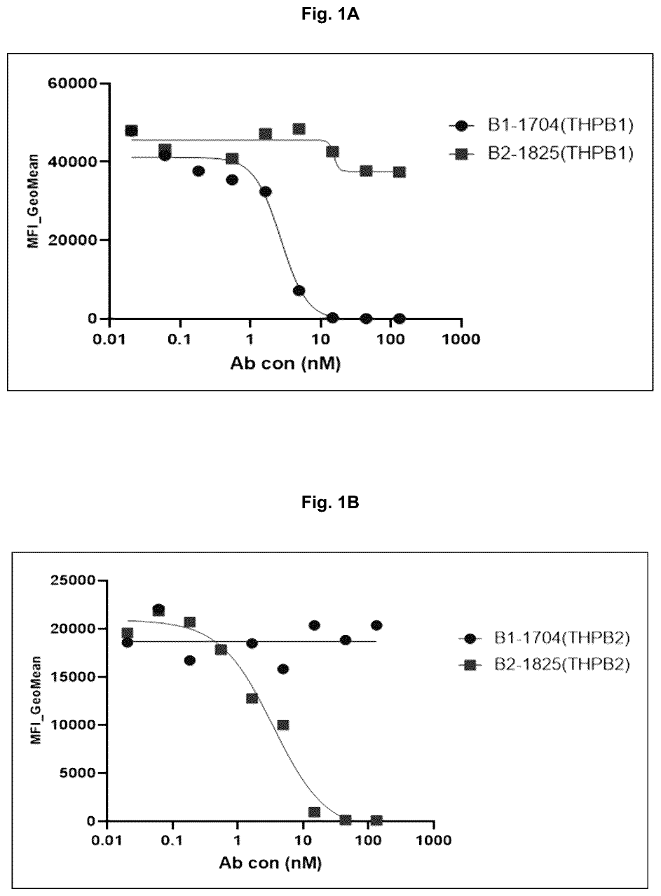

LILRB1 or LILRB2 overexpressing THP1 cells were prepared in FACS buffer (0.5% BSA in PBS) and seeded in 100 μl/well (96 well plate) at 1λ10⁶ cells/ml concentration. The tested antibodies were serially diluted 3-fold into assay buffer starting from 200 nM. 40 μl of serial diluted antibody together with 40 μl of PE labelled HLA-G tetramer (FRED HUTCH IMT40509) at 2 ug/ml were added to each well and incubated on ice for 1 hour. The cells were washed 2 times with FACS buffer and resuspended in 50 μl/well FACS buffer containing 1:1000 dilution of LIVE/Dead fixable violet dead cell stain (Invitrogen #L34964), incubated on ice for 15 minutes. The cells were then washed with FACS buffer for 2 times and resuspended in 80 μl of Cytofix buffer (BD Cat #554655). Analysis was run on Fortessa, anti-huLILRB1 antibody 1704 showed dose-dependent blocking of HLA-G binding to THP1 LILRB1 expressing cells, and IC50 for both antibodies is 1.5 nM. Anti-huLILRB2 antibody 1825 showed dose dependent blocking of HLA-G binding to THP1 LILRB2 expressing cells, and IC50 for 1825 is 1.7-6 nM. No inhibition of HLA-G binding was observed with anti-huLILRB1 Ab_B1-1704 on THP1-LILRB2 cells, or anti-huLILRB2 antibody Ab_B2-1825 on THP1-LILRB1 cells. The results are described in Table 6 and shown in FIG. 1A and FIG. 1B.

TABLE 6
EC50 of HLA-G tetramer and LILRB1
or LILRB2 Interaction Inhibition

| | Anti-LILRB1 antibody | | Anti-LILRB2 antibody | |
|---|---|---|---|---|
| | AB-B1-1704 | | AB-B2-1825 | |
| | THP-B1 | THP-B2 | THP-B1 | THPB2 |
| HLA-G | 2.695 | No competition | No competition | 3.429 |
| Competition | | | | |
| EC50(nM) | | | | |

This Example demonstrates Anti-huLILRB1 antibody Ab_B1-1704 and anti-huLILRB2 antibody Ab_B2-1825 inhibits the interaction between HLA-G tetramer with LILRB1 and LILRB2 expressed on THP1 cells respectively.

### Example 3: Binding Affinity and Specificity of BsAb-1882 Assessed by Surface Plasmon Resonance (SPR) and ELISA

Biacore kinetic assays were conducted at 37° C. with a collection rate of 10 Hz using the Biacore 8K and 8K+instruments (Cytiva). Anti-LILRB2 antibodies were captured by an anti-human IgG (Fc specific) antibody (BR-1008-39, Cytiva) covalently coupled onto a CM5 sensor chip (29-1496-03, Cytiva) according to the manufacturer's instructions. The final capture levels of the antibodies ranged from 20 resonance units (RU) to 60 RU. HBS-EP+pH 7.4 (10 mM HEPES pH 7.4, 150 mM NaCl, 3 mM EDTA, 0.05% Tween 20) was used as sample and running buffer. Flow cell 1 was used as a reference flow cell. Two-fold serial dilutions of human LILRB1 (R&D Systems, Catalog number: 8429-T4) were prepared with concentrations from 405 nM to 15 nM. Dilutions were prepared in duplicate and injected over all flow cells for 60 s at flow rate of 50 μl per minute. Dissociation was monitored for 600 s and the surface was regenerated with two 30 s injections of 3M MgCl₂ at flow rate of 50 μl per minute. The resulting data was double referenced (Myszka, D. G. Improving biosensor analysis. J. Mol. Recognit. 12:279-284; (1999). Rate constants and affinities were determined by fitting the sensorgram data to a Langmuir 1:1 model using Biacore Insight Evaluation software version 3.0.12 (Cytiva). Results are summarized in Table 7.

TABLE 7
Binding Affinities of BsAB-1882 to huLILRB1 and huLILRB2

| | Ligand/ | | | | |
|---|---|---|---|---|---|
| Analyte | antibody | ka (1/Ms) | kd (1/s) | t½ (s) | K_{D} (nM) |
| huLILRB1 | BsAb- | 4.57E+05 | 1.35E−02 | 5.13E+01 | 29.78 ± 1.6 |
| | 1882 | | | | |
| huLILRB2 | BsAb- | 4.43E+05 | 3.23E−04 | 2.15E+03 | 0.73 ± 0 |
| | 1882 | | | | |

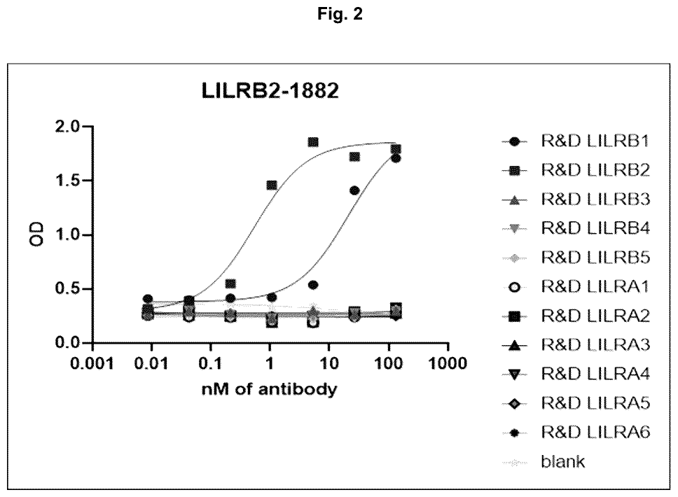

The binding specificity of bispecific antibody BsAb-1882 was determined by ELISA. Purified target proteins huLILRA1, A2, A3, A4, A5, A6, huLILRB1, B2, B3, B4 and B5 (all purchased from R&D) were coated on Nunc-Maxisorb 96-well ELISA plate at 1 ug/ml concentration and stored at 4° C. overnight. Plate was washed 3 times with PBS+0.05% Tween-20 and blocked with PBS+3% milk for 1 hour at room temperature. Tested LILRB1/LILRB2 bispecific antibody was serially diluted 5-fold in PBS+3% milk, starting from 133 nM. Blocking solution was removed and serially diluted antibody was added and incubated for 1 hour at room temperature. Plate was washed and a secondary detecting antibody (goat anti-human IgG-HRP from Invitrogen, Cat #31413, 1:5000) was added and incubated for 30 minutes at room temperature. Plate was washed again for 5 times; signal was developed using TMB substrate and the reaction stopped with 0.18M H₂SO₄. Absorbance was read at 450 nM on an envision plate reader (Perkin Elmer). ELISA result, as shown in FIG. 2, demonstrated LILRB1/LILRB2 bispecific antibody is highly specific to huLILRB1 and huLILRB2. There were no binding activities observed to any of the huLILRA family members tested, and no binding to huLILRB3, B4 or B5.

This Example demonstrated that anti-huLILRB1/2 bispecific antibody BsAb-1882 is highly potent in its binding to huLILRB1 and B2, and it is highly specific and selective in such binding as well as it does not bind to any of the other huLILRA, namely huLILRA1, A2, A3, A4, A5 and A6 and huLILRB members tested, namely huLILRB3, B4 and B5.

### Example 4. Ligand HLA-G Neutralization Assessment of BsAb-1882 and BsAb-1880

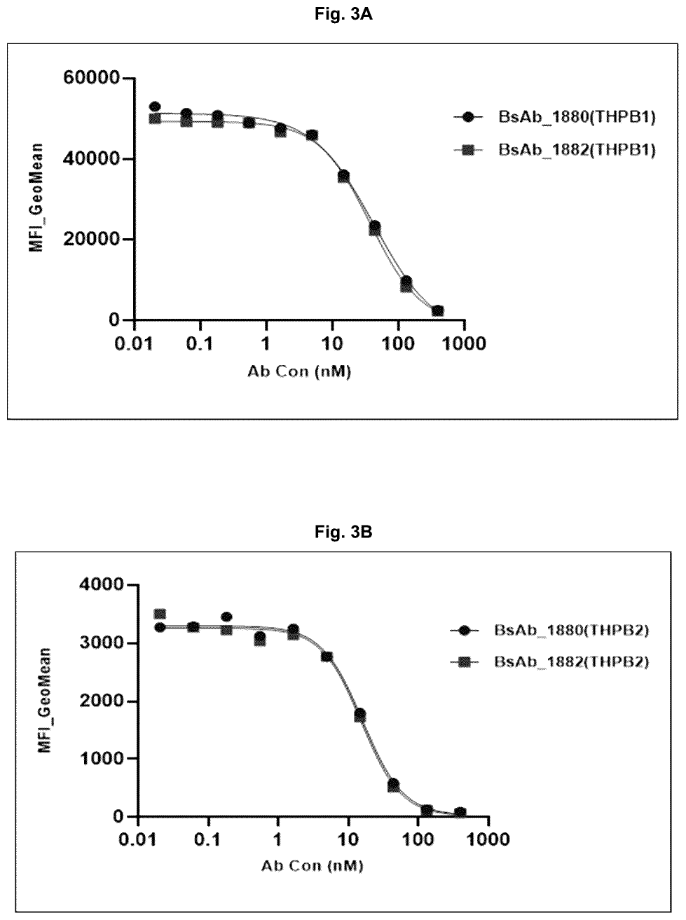

LILRB1×LILRB2 bispecific antibodies BsAb-1882 and BsAb-1880 were tested in cell-based ligand competition assays for their inhibition of the interaction of recombinant HLA-G tetramer with either LILRB1 or LILRB2 expressed on THP1 cells. The cell-based HLA-G ligand blocking assays followed the same procedure as described in Example 2. Both tested bispecific antibodies showed dose-dependent blocking of HLA-G binding to THP1 LILRB1 expressing cells and THP1 LILRB2 expressing cells, as shown in FIG. 3A and FIG. 3B. The IC50 values are summarized in Table 8.

TABLE 8
IC50 of Inhibition of HLA-G Interaction with
LILRB1 and LILRB2 by BsAb-1882 and BsAb-1880

IC50 (nM)

| | LILRB1 | LILRB2 |
|---|---|---|
| BsAb-1882 | 37.6 | 15.4 |
| BsAb-1880 | 43.2 | 16.1 |

Example 4 demonstrated that Bs-1882 and Bs-1880 block interactions between HLA-G and LILRB1 and between HLA-G and LILRB2, with high potencies.

### Example 5. Anti-Tumor Efficacy of BsAb-1882 in Combination with Sasanlimab in Breast Cancer Xenograft

An in vivo tumor efficacy study was carried out in a humanized mouse model with implanted MDA-MB-231 human breast cancer cells to evaluate antitumor efficacy (TGI) of LILRB1×LILRB2 bispecific antibody BsAb-1882.

Neonatal NSG mice were engrafted with human CD34+ hematopoietic stem cells from cord blood at JAXR according to their protocol. Blood of those mice was tested for engraftment of human B, T and myeloid cells by JAX® via flow cytometry approximately 12 weeks after engraftment. MDA-MB-231 cells (5×10⁶) were subcutaneously inoculated into the flank of hCD34+-NSG mice. When tumors reached an average of ˜115 mm³ (21 days after inoculation), mice were randomly allocated to three treatment groups based on tumor size and dosed, starting the following day, subcutaneously (SC) weekly, with control anti-HA antibody only, sasanlimab (anti-PD-1 antibody) plus control anti-HA IgG1 antibody (referred to as sasanlimab single agent); or sasanlimab in combination with BsAb-1882, as shown in below Table 9.

TABLE 9
Treatment Scheme of BsAb and Sasanlimab in Xenograft Mice

| | Mice per | | Dose (mg/ | Route and |
|---|---|---|---|---|
| Group | group | Treatment | kg/dose) | Frequency |
| 1 | 10 | Anti-HA antibody | 30 | SC, one per week |
| 2 | 10 | Sasanlimab | 10 | SC, once per week |
| | | Anti-HA antibody | 20 | SC, once per week |
| 3 | 10 | Sasanlimab | 10 | SC, once per week |
| | | BsAb-1882 | 20 | SC, once per week |

Tumor length and width were measured with a caliper twice weekly and tumor volume was calculated based on the following equation: 1/2 length in mm×width×width. Body weight was also recorded twice weekly. Body weight loss (BWL) of >20% or clinical signs required euthanasia. On day 27 after dosing start, all remaining mice were euthanized.

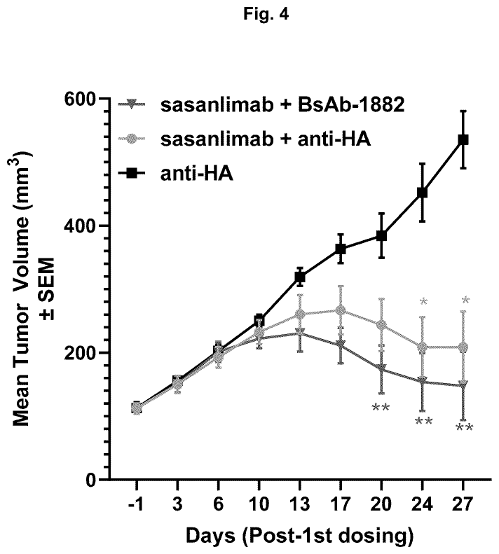

The results are shown in FIG. 4. Mice were dosed on day 0, 7, 14 and 21. On Day 20, both sasanlimab single agent (TGI 51%, p<0.05) and BsAb-1882 in combination with sasanlimab (TGI 78%, p<0.001) significantly decreased tumor growth compared to the control antibody group. Similarly, both sasanlimab single agent and in combination with BsAb-1882 showed statistically significant TGI on Day 24 (71%, p<0.01, and 88%, p<0.001, respectively) and Day 27 (77%, p<0.01, and 92%, p=0.0001, respectively) compared to control antibody group. The BsAb-1882 plus sasanlimab combination group had higher TGI compared to the sasanlimab single agent group throughout the study but did not reach statistical significance.

Example 5 demonstrated that BsAb-1882 shows anti-tumor efficacy in combination with anti-PD-1 antibody, such as sasanlimab, in treating cancer, such as breast cancer.

### Example 6. BsAb-1882 Induced Reversal of HLA-G Mediated Suppression of TNF-α Release from Monocytes and MO Macrophages

Biotinylated anti-CD64 antibody (Biolegend) at a final concentration of 5 μg/mL with or without biotinylated HLA-G (at a final concentration of 3 μg/mL) were coated onto streptavidin high-capacity plates (ThermoFisher Scientific) for 2 hours at room temperature. Plates were then washed twice with PBS before use. Monocytes were negatively isolated from frozen PBMCs using a pan monocyte isolation kit (Miltenyi Biotec) following the manufacturer's instruction. MO macrophages were differentiated from monocytes in the presence of human M-CSF at 50 ng/ml for 7 days. The differentiation of the MO macrophages was confirmed by phase morphology and was immunophenotyped using cell-surface markers by FACS (eg, CD14⁺, CD206⁺, CD163⁺, CD80/86^{low}). Monocytes/MO macrophages were seeded at 150,000 to 250,000 cells/well in Optimem medium (Life Technologies). BsAb-1882 or a-HA as a negative control antibody were added at concentrations ranging from 0.0003-30 g/mL for monocytes and 0.04-30 ug/mL for macrophages in a total volume of 200 μL/well. Supernatants were collected after 20 to 24 hours for detection of TNF-α levels by ELISA according to the manufacturer's instructions (human TNF-α Quantikine ELISA kit, R&D Technologies). Results are shown in Table 10 and Table 11.

TABLE 10
Summary of TNF-a IC₅₀ Values for BsAb-1882 in Monocytes

| PMBC | | IC50 | IC50 | IC50 |
|---|---|---|---|---|
| Donor & lot | Compound/lot | (nM) | (nM) | (nM) |
| 1 | BsAb- 1882/1 | 0.1599 | 0.2 ± 0.0 | 1.2 ± 1.0 |
| | BsAb-1882/2 | 0.2049 | | |
| 2 | BsAb-1882/1 | 2.766 | 3.2 ± 0.6 | |
| | BsAb-1882/3 | 3.639 | | |
| 3 | BsAb-1882/3 | 0.1982 | 0.2 | |

TABLE 11
Summary of TNF-a IC₅₀ Values for
BsAb-1882 in M0 Macrophages

| PMBC | | IC50 | IC50 |
|---|---|---|---|
| Donor & lot | Compound/lot | (nM) | (nM) |
| 4 | BsAb-1882/2 | 15.4 | 28.0 ± 6.4 |
| 5 | BsAb-1882/2 | 32.6 | |
| 6 | BsAb-1882/2 | 35.9 | |

As shown in Tables 10 and 11, incubation with BsAb-1882 reversed HLA-G 5 inhibition of TNF-α release with average IC₅₀ values of 1.2 nM for monocytes and 28.0 nM for macrophages.

Example 6 demonstrated BsAb-1882 reverses HLA-G mediated inhibition of TNF-α production in primary monocytes and macrophages.

1. An isolated bispecific antibody comprising two binding arms, one arm binds to LILRB1 (B1 arm), and the other arm binds to LILRB2 (B2 arm), the B1 arm comprises a B1 arm heavy chain comprising a B1 arm heavy chain variable region (VH) and a B1 arm light chain comprising a B1 arm light chain variable region (VL), and the B2 arm comprises a B2 arm heavy chain comprising a B2 arm VH and a B2 arm light chain comprising a B2 arm VL, wherein
(i) the B1 arm VH comprises a VH CDR1 having the amino acid sequence shown in SEQ ID NO: 9, 10 or 11, a VH CDR2 having the amino acid sequence shown in SEQ ID NO: 12 or 13, and a VH CDR3 having the amino acid sequence shown in SEQ ID NO: 14;
(ii) the B1 arm VL comprises a VL CDR1 having the amino acid sequence shown SEQ ID NO: 2, a VL CDR2 having the amino acid sequence shown in SEQ ID NO: 3, and a VL CDR3 having the amino acid sequence shown in SEQ ID NO: 4;
(iii) the B2 arm VH comprises a VH CDR1 having the amino acid sequence shown in SEQ ID NO: 24, 25 or 26, a VH CDR2 having the amino acid sequence shown in SEQ ID NO: 27 or 28, and a VH CDR3 having the amino acid sequence shown in SEQ ID NO: 29; and
(iv) the B2 arm VL comprises a VL CDR1 having the amino acid sequence shown SEQ ID NO: 18, a VL CDR2 having the amino acid sequence shown in SEQ ID NO: 19, and a VL CDR3 having the amino acid sequence shown in SEQ ID NO: 20.

2. An isolated bispecific antibody comprising two binding arms, one arm binds to LILRB1 (B1 arm), and the other arm binds to LILRB2 (B2 arm), the B1 arm comprises a B1 arm heavy chain comprising a B1 arm heavy chain variable region (VH) and a B1 arm light chain comprising a B1 arm light chain variable region (VL), and the B2 arm comprises a B2 arm heavy chain comprising a B2 arm VH and a B2 arm light chain comprising a B2 arm VL, wherein:
(v) the B1 arm VH comprises the amino acid sequence shown in SEQ ID NO: 16,
(vi) the B1 arm VL comprises the amino acid sequence shown in SEQ ID NO: 6,
(vii) the B2 arm VH comprises the amino acid sequence shown in SEQ ID NO: 31, and
(viii) the B2 arm VL comprises the amino acid sequence shown in SEQ ID NO: 22.

3. The isolated bispecific antibody of claim 1, wherein the bispecific antibody is a human IgG antibody.

4. The bispecific antibody of claim 3, wherein the bispecific antibody is a full length human IgG1 antibody.

5. An isolated bispecific antibody comprising two binding arms, one arm binds to LILRB1 (B1 arm), and the other arm binds to LILRB2 (B2 arm), the B1 arm comprises a B1 arm heavy chain and a B1 arm light chain, and the B2 arm comprises a B2 arm heavy chain and a B2 arm light chain, wherein:
(i) the B1 arm heavy chain comprises the amino acid sequence shown in SEQ ID No 8, and the B1 arm light chain comprises the amino acid sequence shown in SEQ ID No: 1 or SEQ ID NO: 35; and
(ii) the B2 arm heavy chain comprises the amino acid sequence shown in SEQ ID No: 23 or SEQ ID NO: 36, and the B2 arm light chain comprises the amino acid sequence shown in SEQ ID NO: 17.

6. A pharmaceutical composition comprising the bispecific antibody of claim 1.

7. A method to treat cancer in a subject comprising administering to the subject the bispecific antibody of claim 1.

8. The method of claim 7, further comprising administering to the subject an anti-PD-1 antibody.

9. A polynucleotide encoding at least one of the (i) B1 arm VH, (ii) B1 arm VL, (iii) B2 arm VH, and (iv) B2 arm VL, of the bispecific antibody claim 1.

10. A polynucleotide encoding at least one of the (i) B1 arm heavy chain, (ii) B1 arm light chain, (iii) B2 arm heavy chain, and (iv) B2 arm light chain, of the bispecific antibody of claim 1.

11. A vector comprising the polynucleotide of claim 9.

12. A host cell comprising the vector of claim 11.

13. An isolated bispecific antibody comprising two binding arms, one arm binds to LILRB1 (B1 arm), and the other arm binds to LILRB2 (B2 arm), the B1 arm comprises a B1 arm heavy chain comprising a B1 arm heavy chain variable region (VH) and a B1 arm light chain comprising a B1 arm light chain variable region (VL), and the B2 arm comprises a B2 arm heavy chain comprising a B2 arm VH and a B2 arm light chain comprising a B2 arm VL, wherein:
(i) the B1 arm VH comprises the amino acid sequence encoded by the nucleic acid sequence of the insert of the plasmid deposited with ATCC having ATCC Accession No. PTA_127715,
(ii) the B1 arm VL comprises the amino acid sequence encoded by the nucleic acid sequence of the insert of the plasmid deposited with ATCC having ATCC Accession No. PTA_127716,
(iii) the B2 arm VH comprises the amino acid sequence encoded by the nucleic acid sequence of the insert of the plasmid deposited with ATCC having ATCC Accession No. PTA_127717, and
(iv) the B2 arm VL comprises the amino acid sequence encoded by the nucleic acid sequence of the insert of the plasmid deposited with ATCC having ATCC Accession No. PTA_127718.

14. An isolated anti-LILRB1 antibody comprising a heavy chain variable region (VH) and a light chain variable region (VL), wherein:
(i) the VH comprises a VH CDR1 having the amino acid sequence shown in SEQ ID NO: 9, 10 or 11, a VH CDR2 having the amino acid sequence shown in SEQ ID NO: 12 or 13, and a VH CDR3 having the amino acid sequence shown in SEQ ID NO: 14; and
(iii) the VL comprises a VL CDR1 having the amino acid sequence shown SEQ ID NO: 2, a VL CDR2 having the amino acid sequence shown in SEQ ID NO: 3, and a VL CDR3 having the amino acid sequence shown in SEQ ID NO: 4.

15. The isolated anti-LILRB1 antibody of claim 14, comprising a VH that comprises the amino acid sequence shown in SEQ ID NO: 16, and a VL that comprises amino acid sequence shown in SEQ ID NO: 6.

16. A pharmaceutical composition comprising the anti-LILRB1 antibody of claim 14.

17. An isolated polynucleotide encoding the VH, the VL or both the VH and VL of the anti-LILRB1 antibody of any one of claim 14.

18. A vector comprising at least one polynucleotide of claim 17.

19. A host cell comprising the vector of claim 18.

20. A method to treat cancer comprising administering to the subject an anti LILRB1 antibody of claim 14.

21. An isolated anti-LILRB2 antibody comprising a heavy chain variable region (VH) and a light chain variable region (VL), wherein:
(i) VH comprises a VH CDR1 having the amino acid sequence shown in SEQ ID NO: 24, 25 or 26, a VH CDR2 having the amino acid sequence shown in SEQ ID NO: 27 or 28, and a VH CDR3 having the amino acid sequence shown in SEQ ID NO: 29; and
(iii) the VL comprises a VL CDR1 having the amino acid sequence shown SEQ ID NO: 18, a VL CDR2 having the amino acid sequence shown in SEQ ID NO: 19, and a VL CDR3 having the amino acid sequence shown in SEQ ID NO: 20.

22. The isolated anti-LILRB2 antibody of claim 21 comprising a VH that comprises the amino acid sequence shown in SEQ ID NO: 31, and a VL that comprises amino acid sequence shown in SEQ ID NO: 22.

23. A pharmaceutical composition comprising anti-LILRB2 antibody of claim 21.

24. A method to treat cancer in a subject comprising administering to the subject the anti-LILRB2 antibody of claim 21.

25. An isolated polynucleotide encoding the VH, the VL or both the VH and VL of the anti-LILRB2 antibody of claim 21.

26. A vector comprising at least one polynucleotide of claim 25.

27. A host cell comprising the vector of claim 26.

28. The isolated anti-LILRB1 antibody of claim 14 comprising a VH that comprises the amino acid sequence encoded by the nucleic acid sequence of the insert of the plasmid deposited with ATCC having ATCC Accession No. PTA_127715, and a VL that comprises the amino acid sequence encoded by the nucleic acid sequence of the insert of the plasmid deposited with ATCC having ATCC Accession No. PTA_127716.

29. The isolated anti-LILRB2 antibody of claim 21 comprising a VH that comprises the amino acid sequence encoded by the nucleic acid sequence of the insert of the plasmid deposited with ATCC having ATCC Accession No. PTA_127717, and a VL that comprises the amino acid sequence encoded by the nucleic acid sequence of the insert of the plasmid deposited with ATCC having ATCC Accession No. PTA_127718.
